(19) United States
(12) Patent Application Publication
(10) Pub. No.: US 2010/0278784 A1
(43) Pub. Date: Nov. 4, 2010

(54) METHODS AND COMPOSITIONS FOR TREATING SKIN CONDITIONS

(75) Inventors: Kevin Pojasek, Cambridge, MA (US); David Steinberg, Milton, MA (US); Daphne Zohar, Boston, MA (US)

Correspondence Address: CLARK & ELBING LLP, 101 FEDERAL STREET, BOSTON, MA, 02110, US

(73) Assignee: PureTech Ventures, Boston, MA (US)

(21) Appl. No.: 12/599,911

(22) PCT Filed: May 15, 2008
(86) PCT No.: PCT/US08/06224

§ 371 Date: Jul. 14, 2010

Related U.S. Application Data
(60) Provisional application No. 60/930,306, filed on May 15, 2007.
(60) Provisional application No. 60/930,223, filed on May 15, 2007.
(60) Provisional application No. 60/930,182, filed on May 15, 2007.
(60) Provisional application No. 60/930,183, filed on May 15, 2007.

(51) Int. Cl. A61K 35/12; A61P 17/00; A61P 17/10

(52) U.S. Cl. 424/93.7

(57) ABSTRACT

The invention features methods, kits, and compositions for treating aging-related skin conditions (e.g., wrinkles), pigmentation disorders, acne, and scar formation, as well as methods, kits, and compositions for preventing scar formation.

### CROSS-REFERENCE TO RELATED APPLICATIONS

This application claims benefit of U.S. Provisional Application No. 60/930,223, filed 15 May 2007, U.S. Provisional Application No. 60/930,183, filed 15 May 2007, U.S. Provisional Application No. 60/930,182, filed 15 May 2007, and U.S. Provisional Patent Application No. 60/930,306, filed 15 May 2007 which are hereby incorporated by reference.

### BACKGROUND OF THE INVENTION

In general, the invention relates to methods for treating skin related conditions, including aging-related skin conditions such as wrinkles, skin and hair pigmentation disorders, acne, and scar formation, as well as methods of preventing scar formation.

Cutaneous aging is the result of a combination of chronological factors, environmental factors, and hormonal aging. In aged skin, epidermal thinning, associated with reduced numbers of keratinocytes, results from a decrease in epidermal skin cell turnover which may be observed histologically. In addition, a significant flattening of the epidermal-dermal junction alters the transfer of nutrients between the skin layers leading to a further increase in skin frailty. The dermis experiences a decrease in fibroblast number and fibroblast-mediated collagen and elastin production leading to the loss of skin elasticity and the appearance of wrinkles. Skin connective tissue is comprised primarily of fibrillar collagen bundles and elastic fibers. Collagen and elastin impart strength and resilience to skin, and their degeneration with aging causes skin to become fragile, and aged in appearance. Skin aging also produces a reduction in dermal microvasculature also leading to skin atrophy. The skin's subdermal fat tissue is also reduced over time further contributing to skin wrinkles and laxity. Taken together, a decrease in skin thickness and alterations to nutrient distribution lead to the overall decrease in skin elasticity and increase in wrinkling associated with aging skin.

Skin aging is also dramatically impacted by extrinsic, environmental factors such as sun exposure, pollution, harsh weather, and cigarette smoke. Photoaging refers to the additive effects of long term UV exposure on top of the normal skin aging process.

Pigmentation disorders can affect either the skin or the hair leading to an abnormal increase or decrease in the amount of pigment in a given area. Such disorders of abnormal pigmentation in humans include albinism, melasma, vitiligo, hair graying, freckles, hemochromatosis, hemosideriosis, tinea versicolor, and others.

Coloration of the skin and hair is controlled by pigment-producing cells known as melanocytes. Differentiated melanocytes are responsible for integrating melanin into the growing hair shaft. Therapies that can specifically alter hair pigmentation would be a significant improvement over the bleaches and dyes currently available on the market. In addition, melanocytes are found outside of the follicle throughout the dermal layer of skin. Sun exposure leads to an increase in the production of melanin that is, in turn, transferred to the other epithelial cells in the skin. In addition to causing a tan, increased melanin production also reduces UV damage associated with sun exposure and can help reduce the onset of cancer and other severe skin diseases.

Significant research has gone into studying the role of melanocyte biology during embryogenesis. Melanoblasts, the primary melanocyte precursors, arise from the neural crest early in the developing embryo and migrate to their eventual site in the organism, such as the skin's dermis and epidermis as well as the hair follicle. Tyrosinase-related protein 2 (TRP2) and stem cell factor (SCF)/Kit ligand (Kit1) are two well known molecular markers of melanoblast cells. Mitf is a transcription factor that is considered the master regulator of melanocyte function. Mutations in Mitf, along with Pax3 and SOX10, have been linked to different genetic pigmentation disorders. In the adult, melanocyte stem cells are found in the bulge region of the follicle as well as interspersed in the interfollicular epidermis. Controlled stimulation and differentiation of these melanocyte stem cells could be useful for treating a range of skin and hair pigmentation disorders.

Acne arises from a complex combination of abnormal epidermal cell proliferation (known as “hyperkeratinization”), hormonal signaling, bacterial infection, and immune hypersensitivity. This skin disorder occurs in the pilosebaceous follicles composed of the epidermal cells lining the infundibulum, the opening of the follicle, the follicle shaft, and the sebaceous gland. The primary acne lesion is known as a microcomedo, or a blackhead. While the exact cause of microcomedos isn't certain, they are characterized by an impacted and distended follicle with keratinized plug and abnormal sebum production, a complex mixture of lipids that lubricates the follicle and skin.

Acne primarily occurs in pilosebaceous follicles found on the head and upper trunk due to the enhanced sebaceous gland activity compared to follicles found on other parts of the body (e.g., the scalp). Puberty onset generates increased androgen signaling in the skin thus leading to increased sebum production. In addition, to keratinization and altered sebum production, acne lesions are also colonized by Propionibacterium acnes, a fairly inert component of normal skin flora. Within a comedone, P. acnes metabolism of the sebum lipids stimulates the innate immune system through pathways such as neutrophil chemoattraction and complement activation. These general inflammatory stimuli result in the inflamed lesion that assumes the clinical appearance of the typical acne pimple. Acne lesion remission correlates with a down regulation of sebum production and scaling back of the inflammatory response.

In the US alone 40-60 million people are affected with acne, of which 7 million seek prescription treatment and 15 million use over the counter creams. 20 million Americans have acne severe enough to cause scars.

Drugs for the treatment of mild acne are effective in the majority of patients, but treatment options for more severe forms lack sufficient efficacy and often have serious side effects. Mild acne is currently treated with topical compounds that reduce the inflammation of the sebaceous gland including retinoids, benzoyl peroxide, and azelaic acid. The topical retinoids (e.g., Retin-A) are vitamin A derivatives that promote exfoliation of the epithelial cells, thus preventing clogging of the sebaceous gland. Oral agents, including antibiotics and Accutane, are used for the treatment of moderate to severe acne. Accutane is the first line treatment for the most severe form of acne and the most effective therapy for suppressing acne over the long term, but it can have extreme side effects that forced the FDA to create a prescriber and patient registry leading to severely restricted use of the drug.

Several laser-based therapies are currently approved for the treatment of acne. One of the most commonly used treatments is known as photodynamic therapy (PDT). A light-absorbing compound (5-aminolevulinic acid) is applied to the skin followed by laser irradiation. PDT has demonstrated efficacy in 60-75% of patients but has yet to achieve widespread adoption due to the cost of multiple treatments and the extended healing period required post-treatment.

Advances in understanding the molecular components involved in acne pathogenesis will lead to novel therapeutic approaches to this disorder. In addition, new combinations of anti-inflammatory drugs with other compounds that specifically target sebaceous gland biology can provide enhanced relief with reduced side effect profiles for patients suffering from acne. Finally, laser treatments and other device-based approaches offer a new avenue for safely treating and ultimately curing acne.

Scar formation in the adult skin is the result of the incomplete restoration of the cutaneous architecture and strength after injury. Significant research effort has focused on identifying a reproducible way to induce scarless wound healing in adult skin, largely to no avail. Fetal skin during the different stages of embryogenesis is known to heal without significant scar formation. The recent identification of numerous cellular and molecular differences between the embryonic and adult wound healing scenarios offers promise for replicating scarless wound healing in adult skin.

Fetal and adult fibroblasts differ significantly in their ability to generate the extracellular matrix (ECM) constituents that are critical to wound healing. For example, embryonic fibroblasts synthesize more type III and IV collagen than their adult counterparts. This additional collagen synthesis is thought to reduce the infiltration of proinflammatory cells that are known to contribute to scar formation. Embryonic fibroblasts also produce greater amounts of hylauronic acid (HA) and have a higher density of HA receptors on their cell surface leading to an increased ability to migrate throughout the healing wound.

### SUMMARY OF THE INVENTION

In one aspect, the invention features a method of treating a skin related condition selected from an aging related skin condition, a skin and/or hair pigmentation disorder, acne, and scar formation, as well as a method of preventing scar formation in a subject (e.g., a human) by inducing reepithelialization of the skin of the subject and contacting the cells of the skin with a therapeutic compound. The therapeutic compound is administered in an amount sufficient to improve the skin condition.

In another aspect, the invention features a method of treating a skin related condition selected from an aging related skin condition, a skin and/or hair pigmentation disorder, acne, and scar formation, as well as a method of preventing scar formation in a subject (e.g., a human) by contacting the cells of the skin of the subject with a therapeutic compound. In this aspect, the skin is undergoing reepithelialization and the therapeutic compound is administered in an amount sufficient to improve the skin condition.

In yet another aspect, the invention features a method of treating a skin related condition selected from an aging related skin condition, a skin and/or hair pigmentation disorder, acne, and scar formation, as well as a method of preventing scar formation in a subject (e.g., a human) by contacting the cells of the skin of the subject with a therapeutic compound. In this aspect, the skin has been disrupted (e.g., by sub-epidermal or dermal disruption), and the therapeutic compound is administered in an amount sufficient to improve the skin condition.

In any of the foregoing aspects, the reepithelialization can be characterized by an embryonic-like state or by a substantial lack of a stratum corneum. The therapeutic compound selected to improve an aging related skin condition can be a compound that modulates the retinoic acid signaling pathway (e.g., trans-retinoic acid, N-retinoyl-D-glucosamine, and seletinoid G), the estrogen signaling pathway (e.g., 17β-estradiol and selective estrogen receptor modulators), the ubiquitin-proteasome system, or a cytokine signaling (e.g., Imiquimod and IL-1alpha). The therapeutic compound can also be a cell (e.g., a cell capable of inducing differentiation of an uncommitted epidermal cell and a cell capable of differentiating into an epidermal cell).

The therapeutic compound selected to improve a pigmentation disorder can be a compound that modulates a pathway selected from melanocortin signaling, tyrosinase activity, apoptosis signaling, endothelin signaling, nuclear receptor signaling, TGFβ-SMAD signaling, bone morphogenetic protein signaling, stem cell factor signaling, or cytokine signaling. The therapeutic compound can also be a cell (e.g., a cell capable of inducing differentiation of an uncommitted epidermal cell and a cell capable of differentiating into an epidermal cell).

The therapeutic compound selected to improve acne can be a compound that modulates a pathway selected from androgen signaling, retinoic acid signaling, peroxisome proliferator-activated response receptor signaling, estrogen signaling, cytokine signaling, growth factor signaling, nonandrogenic hormone signaling, toll-like receptor signaling, or neurotrophin and neuroendocine signaling. The therapeutic compound can also be a cell (e.g., a cell capable of inducing differentiation of an uncommitted epidermal cell and a cell capable of differentiating into an epidermal cell).

The therapeutic compound selected to improve a scar or prevent formation of a scar can be a compound that modulates a pathway selected from TGF-β signaling, integrin and ECM-mediated signaling, insulin growth factor signaling, cytokine signaling, growth factor signaling, or matrix metalloproteinase signaling. The therapeutic compound can also be a cell (e.g., a cell capable of inducing differentiation of an uncommitted epidermal cell and a cell capable of differentiating into an epidermal cell).

Reepithelialization may include removing the stratum corneum and reepithelialization may be induced by disrupting the epithelial layer. Disruption of the epithelial layer or skin of a subject may in turn be induced by using a device (e.g., sandpaper, a felt wheel, ultrasound, a supersonically accelerated mixture of saline and oxygen, tape-stripping, peels, pumice pads, Scotch-Brite pads, or microneedles). Alternatively, reepithelialization or disruption of the skin may be induced using a chemical (e.g., phenol, trichloracetic acid, or ascorbic acid, or a protease including papain, bromelain, stratum corneum chymotryptic enzyme, trypsin, dispase, or thermolysin), acoustic radiation or electromagnetic radiation (e.g., electroporation). In one aspect, this disruption does not result in disturbance to the stratum corneum or upper epidermis.

In any of the aspects of the invention, the contacting of the cells of the skin with a therapeutic compound may be performed 1, 2, 3, 4, 5, 10, 15, 24, or 48 hours or 1, 2, 3, 4, 5, 6, 7, 10, 14, 21 days, or more, after induction of reepithelialization or disruption of the skin of the subject. Also, in any of the aspects of the invention, the contacting of the cells of the skin with a therapeutic compound may be performed 1, 2, 3, 4, 5, 10, 15, 24, or 48 hours or 1, 2, 3, 4, 5, 6, 7, 10, 14, 21 days, or more prior to the induction of reepithelialization.

By “aging-related skin condition” is meant a condition resulting from intrinsic aging (i.e., chronological aging) as well as extrinsic aging (i.e., resulting from environmental conditions such as photoaging). Examples of such conditions are wrinkles (e.g., fine and coarse wrinkles), brown spots, dyspigmentation, laxity, yellow hue, telangiectasia, leathery appearance, and cutaneous malignancies. Wrinkles and skin laxity are primarily caused by a decrease in the subcutaneous fat layer combined with decreased collagen and elastin synthesis in the dermis. Alterations in skin pigmentation (e.g., brown spots and dyspigmentation) are related to altered melanocyte function and changes in melanin accumulation within basal keratinocytes. Changes in skin blood vessel dilation and distribution contribute to the appearance of telangiectasia and spider veins. Increased skin malignancies are also associated with increased skin aging and generally result from a combination of environmental exposure (i.e., high UV exposure prior to age 18) and genetics. A reduction of sweat gland number and function is another age-related skin condition.

By “pigmentation disorder” is meant a skin or hair condition arising from abnormal skin or hair pigmentation that may but need not be caused by alterations in melanocyte function or viability. Such disorders include abnormal pigmentation in humans such as albinism, melasma, vitiligo, hair graying, freckles, hemochromatosis, hemosideriosis, and tinea versicolor.

By “acne” is meant a skin condition arising from the pilosebaceous unit characterized by hyperkeratinization, P. acnes infection, and abnormal sebum production and that results in a visible skin lesion.

By “improving scar formation” is meant reduced scarring or scarless wound healing of cutaneous injuries. Examples include scars resulting from surgery, skin injury, acne, burns, keloids and other dermatological disorders, stretch marks, skin infections, skin ulcers, and skin tissue grafting. The reduction may be a 5%, 10%, 25%, 50%, 75%, or 100% reduction in scarring.

By “therapeutic compound” is meant a compound that modulates a pathway or system, as used herein, that is involved in a skin condition selected from an aging-related skin condition, a skin or hair pigment disorder, acne, and scar formation

The terms “administration” and “administering” refer to a method of giving a dosage of a pharmaceutical composition to a patient, where the method is, e.g., topical, oral, intravenous, transdermal, subcutaneous, intraperitoneal, or intramuscular. The preferred method of administration can vary depending on various factors, e.g., the components of the pharmaceutical composition or site at which the aging-related skin condition, pigmentation disorder, acne, or scar formation is occurring. In the methods, kits, and compositions of the invention, the administration is, desirably, topical.

By “an amount sufficient” is meant the amount, for example, of a therapeutic compound required to alleviate an aging related skin condition, pigmentation disorder, acne, or scar formation in a subject in comparison to the absence of treatment. The effective amount of the therapeutic compound used to practice the present invention varies depending upon the compound being used, the manner of administration, and the age, body weight, and general health of the subject. Ultimately, the attending physician will decide the appropriate amount and dosage regimen. This amount is referred to as “an amount sufficient.”

By “disruption” is meant a sufficient amount of disturbance to existing hair follicles and the surrounding epidermis and/or dermis to induce an “embryonic-like” state. This embryonic-like state includes the activation, migration, and differentiation of epithelial stem cells from the bulge region of the hair follicle and the interfollicular epidermis. The depth of skin disruption can include in increasing amounts: partial removal of the stratum corneum, complete removal of the stratum corneum, partial removal of the epidermis, complete removal of the epidermis, partial disruption fo the dermis and complete removal of the dermis. Skin disruption can also include disruption of the mid to lower epidermis and/or dermis without any disturbance to the stratum corneum and/or outer epidermis. Different levels of skin disruption can be accomplished by chemical, energetic, mechanical, sound, ultrasound, and/or light based methods.

As used herein, “reepithelialization” refers to the process that occurs during formation of a new epidermis. Tissue undergoing this process can be characterized by cells in an embryonic-like state or by lack of a stratum corneum.

### DETAILED DESCRIPTION

The invention features methods, kits, and compositions for treating skin related conditions including wrinkles and other types of aging-related skin conditions, skin and hair pigmentation disorders, acne, and for treating and preventing scar formation. The methods of the invention can include reepithelialization of the skin tissue prior to administration of a therapeutic compound. Further details of the methods and compositions of the invention are provided below.

### Indications

The invention features methods of treating aging-related skin conditions, pigmentation disorders, acne, and scar formation.

Aging-related skin conditions can be the result of intrinsic aging (i.e., chronological aging) as well as extrinsic aging (i.e., resulting from environmental conditions). Examples of such conditions include wrinkles (e.g., fine and coarse wrinkles), brown spots, dyspigmentation, laxity, yellow hue, telangiectasia, leathery appearance, lentigines, guttate hypomelanosis, solar keratoses, seborrhoeic keratoses, ephelides, actinic lentigo, and cutaneous malignancies.

Skin and hair pigmentation disorders include albinism, melasma, vitiligo, hair graying, freckles, hemochromatosis, hemosideriosis, and tinea versicolor.

The invention also features methods of treating or preventing scar formation as a result of cutaneous injury. Examples include scars resulting from surgery, skin injury, acne, burns, keloids and other dermatological disorders, stretch marks, skin infections, skin ulcers, and skin tissue grafting

### Reepithelialization

Desirably, the compositions of the invention are administered to a subject's skin while the skin is in a state of reepithelialization. Reepithelialization is the process that occurs during formation of a new epidermis after disruption of the skin's intact surface. Epidermal regeneration is characterized by keratinocyte proliferation and migration from the surrounding skin and the migration of epithelial stem cells from the hair follicles in the disrupted area. The reepithelialization process can also be characterized for the purposes of this invention by the presence of cells in an embryonic-like state during epidermal regeneration.

Reepithelialization can be detected through inspection of the new epidermis where covering of the disrupted area by keratinocytes indicates reepithelialization. The presence of keratinocytes can be observed with the naked eye as a white, glossy, shiny surface that gradually covers the open wound. Using a confocal microscope, keratinocytes can also be visualized as a sheet of “cobblestone” like cells. Alternatively, reepithelialization can be detected through measurement of transepidermal water loss (TEWL). TEWL decreases when the epithelial barrier is restored. Confocal scanning laser microscopy and/or optical coherence tomography can also be used to detect the state of reepithelialization, where again the presence of keratinocytes indicates reepithelialization.

Skin undergoing reepithelialization lacks a stratum corneum. The presence of a stratum corneum can be determined though visual inspection, direct observation of papillary blood vessels using a capillary microscope, or through a colorimetric redox reaction of a compound that reacts in the presence of live cells. For example, 0.01% nitrazine yellow applied to the skin will remain yellow if a stratum corneum is present, and will turn greenish brown if not. In another example, 0.01% bromcresol purple applied to the skin will remain yellow if the stratum corneum is present and will turn purple if the stratum corneum is not present.

The area of reepithelialization is, preferably, between 0-2 centimeters (cm) in width (e.g., 1 cm, 1.5 cm, or 2.0 cm) or greater.

In carrying out the present invention, the state of reepithelialization can be induced. Methods of inducing this state include the disruption of the subject's skin at the location where the compounds of the invention will be administered. Disruption may be achieved, for example, through abrasion (e.g., the rubbing or wearing away of skin), or through any method that results in disturbing the intactness of the epidermis or epidermal layer including burning (e.g., by inducing a sunburn) or perforating the epidermis or epidermal layer. The disruption can either result in partial or complete removal of the epidermal layer at the intended location.

The disruption of the epithelial layer can be accomplished, for example, through mechanical, chemical, electromagnetic, or electrical means. Mechanical means include the use of, for example, sandpaper, a felt wheel, ultrasound, a supersonically accelerated mixture of saline and oxygen, tape-stripping, or peels.

Chemical means of disruption of the epidermis can be achieved, for example, using phenol, trichloracetic acid, or ascorbic acid.

Electromagnetic means of disruption of the epidermis can be achieved, for example, by the use of a laser capable of inducing trans-epithelial injury (e.g., a Fraxel laser, a CO₂ laser, or an excimer laser). Disruption can also be achieved through, for example, the use of visible, infrared, ultraviolet, radio, ultrasound, or X-ray irradiation.

Electrical means of disruption of the epidermis can be achieved, for example, through the application of an electrical current or through electroporation.

Any of the previously mentioned means of disruption can be used to induce, for example, a burn, excision, or microdermabrasion.

Optionally, the skin, following epidermal disruption, is free from contact for a period of time with any substance (e.g., ointment, a bandage, or device) that is normally administered to an abrasion or wound to prevent infection. By this method, the skin is not contacted with any substance until, for example, the epidermal disruption has healed (e.g., any time between 2 days and 3 weeks).

Prior to disruption, the skin can be depilated or epilated. The depilation or epilation can be accomplished through, for example, waxing, plucking, an abrasive material, a laser, electrolysis, a mechanical device, or thioglycolic acid.

The disruption of the epidermis can be induced, for example, 1, 2, 3, 4, 5, 10, 15, 24, or 48 hours or 1, 2, 3, 4, 5, 6, 7, 10, 14, 21 days prior to the administration of the compositions of the invention

### Compounds to be Administered During Reepithelialization to Treat Aging Related Skin Conditions

Following induction of reepithelialization, therapeutic compounds may be applied to the skin according to the methods of the invention. Such therapeutic compounds are, for example, compounds known to alleviate aging related skin conditions and compounds known to modulate signaling pathways associated with such conditions (e.g., the pathways described below).

TGFβ-SMAD and Bone Morphogenetic Protein Pathways

In one embodiment, the invention features administration of compounds that modulate the TGFβ-SMAD signaling pathway and the bone morphogenetic protein (BMP) pathways. BMPs are secreted proteins that broadly regulate cell proliferation, differentiation, and apoptosis through the interaction with and downstream signaling through BMP receptors. Biochemical analysis of BMP-mediated signaling suggests that BMPs interact with other protein families including Wnt, Shh, TGF-β, EGF, FGF, Notch, and others.

During development, BMP-6 is expressed primarily in the suprabasal layers of the epidermis, and BMP-7 is found primarily basal layer of the epidermis. Expression of BMP-2 and BMP-4 is restricted primarily to the developing hair follicle. BMP receptors-IA and BMP receptor-IB are restricted to suprabasal keratinocytes. Smad1, Smad5, Smad6, and Smad7, downstream signaling molecules in the BMP pathway, are also expressed in the developing epidermis.

During embryonic hair follicle induction, BMP-2 and BMPR-IA are found in the hair placode while BMP-4 and noggin, an endogenous inhibitor of BMP signaling, are seen in the mesenchymal cell layer below the thickening epidermis. BMP-2 signaling is also implicated in dermal remodeling, potentially via an interaction with the matrix metalloproteinases (MMP) family of extracellular matrix (ECM)-degrading enzymes.

Modulation of molecular signaling in embryonic and adult skin are commonly mediated through the TGFβ-SMAD pathway. One of the major downstream pathways is the synthesis of collagen 1, the primary collagen in adult dermis. Sphingosine 1-phosphaste and asiaticoside are naturally occurring molecules that can enhance collagen production via TGFβ-SMAD signaling (Lee J. et al. (2006) Planta Med 72:324-28 and Cuidan X. et al. (2004) JBC 279:35255-62).

Compounds useful for the modulation of the TGFβ-SMAD and BMP pathways in conjunction with reepithelialization include, without limitation: Eptotermin alfa, Noggin, bone morphogenetic protein activators (Curis/Ortho Biotech), Transforming growth factor-beta-3, Transforming growth factor-beta-1, Transforming growth factor-alpha, Cetermin, Tamoxifen methiodide, Decorin, Kahalalide F, Anti-TGF-beta monoclonal antibody 2G7, ADMP 1, Lerdelimumab, Metelimumab, TGF-beta antagonists (GLYCODesign), A 161906, LF 984, Tetrathiomolybdate, Tranilast, GC 1008, SEK 1005, TGF-beta antagonists (Scios), SR2F, Stamulumab, NeuGene antisense compounds (AVI BioPharma), TJN 598, TGF-beta RI kinase inhibitors (Scios), TGF-beta oligonucleotide nanoparticles (NanoDel), TGF-beta type I receptor inhibitors (In2Gen), TG-C, and Mannose 6 phosphate.

Reactive-Oxygen Species/Antioxidants

In another embodiment, the invention features administration of compounds that reduce the generation of reactive oxygen species (ROS). Environmental exposure can lead to generation of ROS in the skin. While the skin is equipped with enzymatic and non-enzymatic antioxidants to reduce ROS-mediated cell damage, these pathways can be overwhelmed as the skin ages.

Compounds and enzymes useful for the reducing ROS-mediated skin damage in conjunction with reepithelialization include, without limitation, vitamin E, vitamin C, coenzyme Q₁₀, ascorbate, selenium, proanthocyanidin, α-lipoic acid, cartenoids, soy-isoflavones, genistein, N-acetyl cysteine, gluconolactone, green tea polyphenols, N-furfuryladenine (kinetin), dietary lutein, pine tree extract, superoxide dismutase, catalase, thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and malic acid, humic acid, bile acid, bile extracts, bilirubin, biliverdin, EDTA, EGTA, and DTPA), vitamins (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and glutathione peroxidase.

Retinoic Acid Pathway

In another embodiment, the invention features administration of compounds that modulate the retinoic acid signaling pathway. This pathway has been associated with wrinkles and other aging-related skin conditions (Cho S et al. (2005) J Amer Acad Dermatol 53:769-94). Topical application of retinoic acid has been shown to induce collagen synthesis and enhance elastin function thereby reducing or reversing the effects of aging on the skin.

Compounds useful for the modulation of the retinoic acid signaling pathway in conjunction with reepithelialization include, without limitation, trans-retinoic acid, N-retinoyl-D-glucosamine, seletinoid G, Fenretinide, Liarozole, Tazarotene, AM 580, Bexarotene, Alitretinoin, AR 623, AGN 191701, SR 11237, CGP 52608, LG 100153, LGD 1550, LG 100567, AGN 193835, AGN 193836, MX 33501, MX 28701, NIX 901, MDI 403, LGD 1324, AGN 194310, CD 437, UAB 8, CD 1599, TAC 101, SR 11383, LGD 1268, 4-Oxoretinol, ER 35794, BMS 185411, RO 415253, ER 38925, ER 65250, R 116010, BMS 292974, UAB 30, VN/14-1RA, BMS 297208, LG 101506, Tretinoin, L 007, Isotretinoin, PLT 99511, AGN 195183, AGN 194204, R 667, retinoid X receptor alpha agonists, retinoic acid receptor gamma agonists (Locus), BMS 189453, retinoic acid metabolising enzyme blockers (Bioenvision), LXS/4-HPR, Seletinoid G, Rambazole, fenretinide, and Carbenoxolone.

Peroxisome Proliferator-Activated Response Receptors

In another embodiment, the invention features administration of compounds that modulate peroxisome proliferator-activated response receptor (PPAR) family. PPARs are nuclear hormone, ligand-induced transcription factors that generally act as cellular sensors of polyunsaturated fatty acids and other fatty acid derivatives. PPARα and PPARβ are both expressed at different times and areas of embryonic and adult skin. PPARα is expressed in the adult skin after injury and plays an important role in mediating the initial inflammatory-mediated healing response. PPARα and PPARβ are both constitutively expressed in the hair follicle where they are thought to play an active role in mediating the hair follicle cycle. In addition to its role in hair follicle development, PPARβ also plays a critical role in mediating skin repair in response to injury.

Compounds useful for the modulation of the PPAR signaling pathway in conjunction with reepithelialization include, without limitation, Troglitazone, Pioglitazone, Englitazone, AY 31637, Darglitazone, Rosiglitazone, Ciglitazone, AD 5075, Bexarotene, Netoglitazone, BM 131246, BM 501050, Farglitazar, Balaglitazone, Reglitazar, GW 2570, GW 409890,Tesaglitazar, MK 0767, PD 72953, Ragaglitazar, GW 409544, Rivoglitazone, GW 1929, GW 9578, GW 0072, SB 219994, LG 101506, Metaglidasen, CLX 0921, LR 90, LY 510929, GW 501516, Naveglitazar, NC 2100, PPAR gamma antagonists (Bayer/GSK), LF 200337, GW 5393, PPAR alpha/gamma agonists (Eli Lilly), Muraglitazar, ARH 049020, MBX 2044, KT 6207, GW 7282, PPAR alpha/gamma agonists (Bayer), GW 590735, BAY 549801, L 764406, CLX 0940, NS 220, PPAR gamma agonists (Vita), Fenofibrate, 677954, LY 518674, AMG 131, KRP 101, PPAR agonists (Merck & Co), DRF 4832, ONO 5129, Fenofibrate/metformin, Oxeglitazar, PPAR agonists (GlaxoSmithKline), TY 51501, AA 10090, peroxisome proliferator-activated receptor agonists (Karo Bio), PPAR modulators (Fournier Pharma), AZD 6610, 641597, PPAR delta agonists (Nippon Chemiphar/Pfizer), PPAR pan agonists (Plexxikon), DRF 10945, AVE 0847, PPAR-gamma agonists (Daiichi Sankyo), peroxisome proliferation activated receptor beta modulators (7TM Pharma), Peliglitazar, PPAR alpha agonists (CrystalGenomics, MaxoCore Pharmaceuticals,), PPAR alpha/gamma agonists (MaxoCore Pharmaceuticals), AVE 8134, peroxisome proliferator-activated receptor agonists (Novo Nordisk), PPAR delta agonists (Eli Lilly, Nippon Chemiphar/Cerenis), E 3030, PPAR agonists (Metabolex), DRL 11605, LBM 642, peroxisome proliferator-activated receptor alpha/gamma agonists (sanofi-aventis), PLX 204, peroxisome proliferator-activated receptor modulators (AbGenomics), Fenofibrate/simvastatin, 625019, CS 7017, CKD 501, AVE 5376, PPAR delta agonists (sanofi-aventis), Ezetimibe/fenofibrate, RWJ 800025, Fenofibrate/rosuvastatin calcium, AB 335/rosuvastatin calcium, Fenofibrate/rosuvastatin, Pioglitazone/TAK 536, CDT-fenofibrate, PPAR agonists (Bayer), peroxisome proliferator-activated receptor agonists (Eli Lilly), KD 3010, and GFT 505.

Integrin-Mediated Signaling

In another embodiment, the invention features administration of compounds that modulate integrin-mediated signaling. Integrins are heterodimeric transmembrane receptors composed of an α and β subunit. The most prominent constitutively expressed integrins in the adult epidermis include α2β1 (collagen receptor), α3β1 (laminin 5 receptor), α6β4 (laminin receptor), and αvβ5 (vitronectin receptor). Additional integrins, namely α5β1 (fibronectin receptor), αvβ6 (fibronectin and tenascin receptor), and α9β1 (tenascin receptor) are expressed in response to skin damage and wound healing. In normal skin, integrins are primarily expressed in the basal layer and the hair follicle outer root sheath. Interfollicular and hair follicle stem cells are also known to express the highest levels of β1 integrin, a molecular signature that is often used to identify and enrich epithelial stem cells.

Compounds useful for the modulation of the integrin-mediated signaling pathways in conjunction with reepithelialization include, without limitation, Applaggin, Kistrin, RO 435054, MK 852, G 4120, SC 49992, TP 9201, Eptifibatide, Tirofiban, Anti-CD18 monoclonal antibody, Abciximab, Anti-VLA-4 monoclonal antibody PS/2, Lefradafiban, SKF 107260, DU 728, Lamifiban, CGH 400, SC 52012, GR 91669, SKF 106760, Tetrafibricin, Xemilofiban, Lotrafiban, SB 208651, L 703014, MEDI 522, RWJ 50042, Halystatin, C 6822, SDZ GPI 562, TAK 029, SB 1, L 709780, Fradafiban, SB 6, GR 83895, YM 207, BMW 98, RG 13965, EF 5077, YM 337, Contortrostatin, RWJ 50228, DMP 757, Rovelizumab, SB 207448, SC 56929, L 734217, Disagregin, G 7453, RO 438857, G 5598, RPR 110173, S 1197, ZD 2486, S 1762, FK 633, CY 9652, RO 443888, Sibrafiban, Natalizumab, Roxifiban, XR 300, NSL 9403, L 748415, ME 3277, P 246, TBC 772, RWJ 50271, SC 56631, TRM 147, PS 028, Orbofiban, Alnidofibatide, USB IPA 1302, Monoclonal antibody PMA5, Monoclonal antibody AZ1, MA 16N7C2, RP 431, SB 223245, L 703801, DMP 802, BIO 1050, BIO 1272, L 738167, SR 121566, XU 063, SR 121787, MS 180, MS 28168, ME 3229, integrin antagonists (Integra LifeSciences), Alpha D modulator, Cilengitide, ZD 7349, MLN 0002, T 250, SB 236392, Doxorubicin peptide conjugate, XR 299, integrin antagonists (Celltech), SB 265123, XV 454, MLN 2201, L 734115, SH 306, Cromafiban, TS 963, TS 943, Accutin, Elarofiban, UR 12947, Gantofiban, GR 233548, SM 20302, alphaV-beta3 receptor antagonists (Shire), NSL 96184, SC 68448, FR 158999, S 137, SM 256, integrin antagonists (SIDR), XJ 735, SQ 885, UR 3216, TR 9109, TR 14035, TR 14531, CP 4632, SC 72115, XU 065, VLA-4 antagonists (Biogen/Merck), CT 5219, SB 273005, L 750034, VLA-4 antagonists (Elan/Wyeth), CP 4685, TBC 3486, TBC 3342, ME 3230, RBx 4638, XT 199, VO 514, SB 267268, IVL 745, AR 0510, AR 0598, LFA-1 antagonists (ICOS), integrin receptor antagonists (Johnson & Johnson), ER 68203, Anti-VLA-4 monoclonal antibody HP1/2, Anti-VLA-4 monoclonal antibody TA-2, Anti-VLA-4 monoclonal antibody R1-2, S 787, CT 747, CT 757, CT 767, L 806978, integrin antagonists (Merck & Co), SC 65811, SJ 874, TBC 4257, IC 747, Integrin antagonist (Bayer), VCAM/VLA-4 antagonists (Wyeth), S 247, BIRT 0377, VLA-1 inhibitor (Biogen Idec), VCAM/VLA-4 antagonists (Kaken), RP 593, HMR 1794, TAK 024, Integrin antagonists (Sigma Tau), 559090, vitronectin receptor antagonists (Uriach), VLA-4 antagonists (Uriach), Valategrast, R 1295, integrin antagonists (Targesome), alpha-6 integrin antagonists (Dyax), biologically active linear polysaccharides (BioTie Therapies), TBC 4746, LFA-1 antagonists (Tanabe Seiyaku), RBx 7796, Volociximab, CDP 323, F 200, T 0047, CNTO 95, alpha 2 beta 1 integrin inhibitors (BioTie Therapies), E 7820, BIO 5192, PS 460644, DW 908e, integrin inhibitors (Jerini), integrin avBeta3 inhibitors (Nuevolution), R 1541, Lymphocyte function-associated antigen-1 antagonist (Bristol-Myers Squibb), LFA-1 antagonists (Boehringer Ingelheim), TBC 3804, anti-alpha-5 beta-1 integrin antibody (Pfizer), integrin receptor antagonists (Johnson & Johnson), anti-alpha-v beta-6 monoclonal antibodies (Biogen Idec), and alpha 4 integrin antagonists (Elan).

Estrogen Signaling

In yet another embodiment, the invention features administration of compounds that modulate estrogen signaling. Estrogens are known to prevent skin aging by increasing the thickness of the epidermis, reducing skin wrinkling, and modulating skin moisture. Women have a steady deterioration in their skin architecture after menopause which can be reversed by hormone replacement therapy (HRT). Topical application of 17β-estradiol has been shown to mimic these effects without the peripheral side effects of hormone replacement therapy (HRT) (Verdier-Sevrain et al. (2006) Exp Dermatol 15:83-94 and Son ED et al. (2005) JID 124: 1149-61).

Compounds useful for the modulation of estrogen signaling pathways in conjunction with reepithelialization include, without limitation, 17β-estradiol, estriol, estrone, conjugated estrogens (e.g., Premarin, PremPro), diethylstilbestrol selective ER modulators (SERMS) (e.g., tamoxifen, raloxifene, toremifene, clomifene, bazedoxifene, lasofoxifene, and ormeloxifene), Fulvestrant, ICI 164384, Zindoxifene, Panomifene, CB 7386, RU 39411, LY 133314, RU 58668, ZK 119010, EMATE, Prolame, WS 7528, RU 16117, Yuehchukene, 3-Methyl-3-hydroxy-chalcone, Tesmilifene, RU 45144, CDRI 85287, Tamoxifen methiodide, Estradiol/trimegestone, ICZ, EM 219, Ethinylestradiol/gestodene monophasic, Ethinylestradiol/drospirenone, Complex K, ZK 115194, Ethinylestradiol/dienogest, J 893, BE 25327, Estradiol valerate/dienogest, TS 17, Abarelix, Estradiol/norethisterone, Estradiol/levonorgestrel, Ethinylestradiol/gestodene-triphasic, Centchroman, TS 33, EM 800, Estradiol/nomegestrol, SR 90067, OSW-1, K 7, Anordrin, Ospemifene, Alpha-Fetoprotein, Estradiol/testosterone, IP 1162, IP 1163, IP 1164, J 995, estrogen receptor-alpha antagonists (Sumitomo), Estradiol/norethisterone, Ethinylestradiol/desogestrel, Estradiol cipionate/medroxyprogesterone, Ethinylestradiol/levonorgestrel, Ethinylestradiol/norethisterone, Esterified estrogens, Ethinylestradiol/levonorgestrel, Ethinylestradiol/norethisterone, Ethinylestradiol/chlormadinone, Conjugated estrogens, Estradiol/dydrogesterone, Trilostane, Ethinylestradiol/etonogestrel, P 081, Ethinylestradiol/norgestimate, EMM 210525, Estradiol acetate vaginal, Estradiol/progestogen, SM 16896, Acolbifene, Estradiol valerate/medroxyprogesterone, Ethinylestradiol/gestodene, NNC 450095, SDN 289, TZN 13, BAY 509062, MCC 565, NV 50, Estradiol/nomegestrol, EMM 310525, PSK 3987, S 401G, pure estrogen receptor antagonists (ProStrakan), E2CDS, J 811, J 861, Afimoxifene, Enclomifene, Estradiol/dienogest, BN 83495, SIM 916, ERB 196, Tamoxifen, SIM 688, AP 1081, estrogen-related receptor alpha modulators (Phenex Pharmaceuticals), ORG 43228, and 8-Prenylnaringenin.

Ubiquitin-Proteasome System

In another embodiment, the invention features administration of compounds that modulate the ubiquitin-proteasome system. The ubiquitin-proteasome system (UPS) controls the degradation of cellular proteins and is closely tied to cellular senescence, a critical component of aging skin. In addition, the UPS modulates TGFβ-SMAD and NF-κB signaling, other key pathways in skin aging (Bregegere F et al. (2006) Age Res Rev 5:60-90).

Compounds useful for the modulation of the ubiquitin-proteasome system in conjunction with reepithelialization include, without limitation, Bortezomib, ubiquitin-proteasome inhibitors (Aventis/Millennium), MLN 519, MG 132, CVT 634, TMC 96, TMC 86A, TMC 86B, LCS 640, ubiquitin-proteasome inhibitors (Millennium/Roche), proteasome inhibitors (Millennium), 20S proteasome inhibitors (Novartis), CEP 1612, proteasome inhibitors (Cell Therapeutics/Cephalon), NPI 0052, proteasome inhibitors (Eisai), PR 171, 26S proteasome inhibitors (Ergon Pharmaceuticals), E-3 ubiquitin ligase inhibitors (Rigel), ligase-targeted therapies (Celgene), ubiquitin ligase inhibitors (Proteologics), and ubiquitin specific protease inhibitors (Hybrigenics).

Cytokine and Growth Factor Signaling

In yet another embodiment, the invention features administration of compounds that modulate cytokine and growth factor signaling. Pro-inflammatory cytokines, including IL-1, TNF-α, IL-6, and interferon γ and α, among others, are up-regulated in response to skin damage. Modulation of the cytokines are thought to contribute to the breakdown of the skin function in photoaging (Barland O. et al. (2004) JID 122:330-6). The expression of the genes that code for the proinflammatory cytokines is upregulated in response to via nuclear factor kappaB (NF-kappaB) and AP-1, well known proinflammatory transcription factors. Therefore, stimuli that induce up-regulation of the NF-kappaB pathway contribute to the alteration of the levels of proinflammatory cytokines and therefore skin aging.

Numerous growth factors, including but not limited to members of the fibroblast growth factor (FGF) family (including keratinocytes growth factor), hepatocyte growth factor, and platelet-derived growth factor (PDGF) are also altered in aging skin. For example, UV exposure leads to the NF-kappaB mediated up-regulation of FGF-2, which in turn impacts the proliferation of skin keratinocytes and melanocytes.

Compounds useful for the modulation of cytokine and growth factor signaling in conjunction with reepithelialization include, without limitation, Imiquimod/Avara, IL-1alpha, parthenolide, magnolia extract, magnolol, Prasterone, Iguratimod, Suplatast tosilate, Bindarit, Liarozole, UK 122802, ONO 4007, Stiripentol, DUP 983, DUP 630, DMXAA, ICZ, FPP 33, PP 33, Mesoporphyrin, Semapimod, A 802715, Pirfenidone, Sho-seiryu-to, FR 167653, Pentoxifylline, Iboctadekin, Pimecrolimus, Temsirolimus, REP 689, R 116010, Tadekinig alfa, Prasterone, PB 007, anti-interleukin-18 monoclonal antibodies (CAT), ISIS 104838, Delmitide, P450RAI inhibitors (Cytochroma), ZNC 2381, R 115866, CLX 0921, Thymosin beta-4, M 50367, JTE 607, Licochalcone A, vitamin D signal amplifiers (Cytochroma), TS 011, CF 101, Prasterone phosphocholine, Y 39041, RDP 58 analogues (Genzyme/Synt:em), NPI 1302a-3, AVI 4557, Susalimod, p38 MAP kinase inhibitors (Uriach/Organon), MT 201, interleukin-4/5 secretion inhibitors (Fournier/Zambon), LMP 160, LMP 420, PLR 14, AD-GL0001, CLX 090717, CLX 090502, CRX 102, Ciclosporin, ABT 325, IMS, K 832, CC 10004, Interleukin 6 inhibitor (Y′s Therapeutics), YSTH2, CR1 (Nuada), CRX 119, CRX 139, Golimumab, Rambazole, cytokine receptor antagonists (Trillium Therapeutics), high mobility group box chromosomal protein-1 inhibitors (Nautilus/Creabilis), CYT 007, TNFQb, QR 440, CTA 018, K 412, AN 0128, CRX 170, CRx 140, CRX 150, RC 8800, tumour necrosis factor gene therapy (Onc Bio), Recombinant IL-18 binding protein, HMPL 004, tpl2 kinase inhibitors, SPC 839, RTA 401, MPC 7869, INDRA compounds (Active Biotech), APC 0576, NF-kappa-B Decoy oligonucleotide (Anesiva), BG 12, NF-kappa B/IKK2 inhibitors (Uriach), Antisense oligonucleotide NF-KappaB-p65 (Serono/InDex Pharmaceuticals), I-kappa B kinase inhibitors (Millennium Pharmaceuticals), NeuGene antisense compounds (AVI BioPharma), SIM 916, liposomal calagualine (Plantacor), NF-kappa B inhibitors (Serenex, Scottish Biomedical), SIM 688, NFkappaB pathway inhibitors (4SC), Synthetic triterpenoids (Reata Pharmaceuticals), and RTA 402.

Toll-Like Receptor Signaling

In yet another embodiment, the invention features administration of compounds that modulate toll-like receptor (TLR) signaling. The TLR family of cell surface receptors includes 10 known family members in humans generally involved in pathogen recognition and innate immune system simulation. Several studies identified differential TLR 1, 2, 4, 5, and 9 expression in human keratinocytes at different levels of the skin. Alteration in normal TLR expression has been associated with a variety of human skin diseases and disorders, including leprosy, acne, and psoriasis.

Compounds useful for the modulation of TLR signaling in conjunction with reepithelialization include, without limitation, OM 174, CpG 7909, Eritoran, Isatoribine, toll-like receptor 9 agonists (Idera Pharmaceuticals), IMO 2055, CpG 10101, toll-like receptor 4 modulators (GlaxoSmithKline), toll-like receptor 7/8 agonists (Idera Pharmaceuticals), TLR9 agonists (Coley/sanofi-aventis), CRX 675, TLR9 antagonists (Coley), next-generation toll-like receptor 9 agonists (Coley/Pfizer), Sotirimod, toll-like receptor 3 agonists (Innate Pharma), and toll-like receptor 9 agonists (AstraZeneca/Dynavax).

Matrix Metalloproteinases

In yet another embodiment, the invention features administration of compounds that modulate matrix metalloproteinase (MMP) activity in the skin. The MMP family is composed of 28 members of metal dependent enzymes that break down different extracellular matrix (ECM) components in the body. MMP-1, −3, and −9 are responsible for degrading collagen in human dermis and are up-regulated in response to UV exposure. This UV-induced MMP activity results in reduced collagen levels, a key underlying factor in photoaging of the skin.

Compounds useful for reducing MMP activity in conjunction with reepithelialization include, without limitation zinc chelators, iron chelators, doxycycline, marimastat, trocade, TIMP-1, TIMP-2, TIMP-3, TIMP-4, RO 314724, Ilomastat, Incyclinide, D 1927, SE 205, MMP inhibitors (Millennium), macrophage metalloelastase inhibitors (Novartis), PCK 3145, BB 2827, Apratastat, ONO 4817, matrix metalloproteinase inhibitors (Procter & Gamble), ABT 518, SC 77964, SC 276, matrix metalloproteinase inhibitors (Pfizer), SI 27, MPC 2130, GW 3333, matrix metalloproteinase inhibitors (Cengent Therapeutics/De Novo), matrix metalloprotease inhibitors (LEO Pharma), matrix metalloproteinase inhibitors (Shionogi), RO 282653, S 3536, MMP-12 inhibitor (Serono), TMI 1, dual tumour necrosis factor/matrix metalloproteinase inhibitors (Roche), protease inhibitors (Biopharmacopae), matrix metalloproteinase-13 inhibitors (Alantos), matrix metalloproteinase-13 inhibitors (Wyeth), and matrix metalloproteinase antibodies (Dyax).

Neurotrophin Signaling

In yet another embodiment, the invention features administration of compounds that modulate neurotrophin (NT) signaling in the skin. The neurotrophin family is composed of nerve growth factor (NGF), brain-derived growth factor (BDNF), neurotrophin-3 (NT-3) and neurotrophin-4 (NT-4). High affinity NT receptors belong to the tyrosine kinase family and include TrkA, TrkB, and TrkC. The NT's also interact with p75NTR albeit with a lower affinity. NGF, NT-3, and BDNF are expressed primarily by fibroblasts, although expression has also been noted in cutaneous nerve fibers and myocytes in the arrector pili and panniculus carnosus muscles. Proliferating human keratinocytes produce and secrete NGF. TrkA and TrkB are primarily expressed on epidermal keratinocytes whereas TrkC is found on cutaneous nerve cells and in the hair follicle.

The onset of NT expression is observed early in murine embryonic development in the skin epithelium and mesenchyme, and correlates with epidermal K5 and K14 expression. Maximal embryonic expression correlates with hair follicle induction in murine dorsal skin. NT's also play a critical role in melanoblast and melanocyte migration, viability and differentiation during embryogenesis.

Compounds useful for the modulation of neurotrophin signaling in conjunction with reepithelialization include, without limitation, phorbol 12-tetra decanoate 13 acetate.

Compounds to be Administered During Reepithelialization to Treat Pigmentation Disorders

Following induction of reepithelialization, therapeutic compounds may be applied to the skin according to the methods of the invention. Such therapeutic compounds are, for example, compounds known to treat pigmentation disorders and compounds known to modulate signaling pathways associated with pigmentation disorders (e.g., the pathways described below).

Melanocortin Signaling Pathways

In one embodiment, the invention features administration of compounds that modulate melanocortin signaling pathways. Melanocortins are structurally related peptides that regulate pigmentation of the skin and hair. Naturally occurring melanocortins are derived from selective enzymatic processing of propiomelanocortin (POMC) and include ACTH and the melanocyte stimulating hormones (α-MSH, β-MSH, and γ-MSH). Most cell types in the skin produce melanocortins and express melanocortin receptors, composed of 5 members of G protein coupled protein receptors (named MC-1R through MC-5R).

There is strong evidence implicating the melanocortin pathway in the regulation of cutaneous pigmentation. For example, exogenous systemic administration of α-MSH or other melanocortins leads to an observable increase in skin pigmentation. In addition, patients with POMC null mutations or specific mutations in MC-1R have red hair and altered skin pigmentation. Melanocortin signaling is also induced in response to inflammation and UV light exposure.

Compounds useful for the modulation of the melanocortin signaling pathway in conjunction with reepithelialization include, without limitation, MIF 1, CUV 1647, HP 228, Nemifitide, PT 14, RO 273225, melanocortin-4 receptor antagonists (Gene Logic), melanocortin-4 receptor agonists (Pharmacopeia), melanocortin-4 receptor modulators (Neurocrine Biosciences), Bremelanotide, melanocortin-4 receptor agonists (LION bioscience/Novartis), melanocortin-4 receptor agonists (Melacure Therapeutics), TRG 2411, ZYC 200, melanocortin-4 receptor agonists (Merck), CZEN 002, melanocyte stimulating hormone analogues (Zengen), melanocortin receptor antagonists (Taisho), melanocortin-4 receptor antagonists (Santhera Pharmaceuticals), melanocortin-4 receptor agonists (Palatin Technologies, Novo Nordisk, Amgen, Ipsen, LG Life Sciences, Amura, Eli Lilly, AnaMar Medical), CZEN 003, melanocortin-4 receptor modulators (TransTech Pharma), melanocortin receptor antagonists (Palatin Technologies), AP 214, and RO 0282425.

Tyrosinase Activity

In another embodiment, the invention features administration of compounds that modulate tyrosinase expression, stability, and activity. Melanin production is tightly controlled by an enzyme called tyrosinase; a membrane bound, copper-containing glycoprotein that is the rate limiting step in melanin synthesis. Other important enzymes in the melanin-producing pathway include Dct and Tyrp1. Tyrosinase is expressed solely in melanocytes and leads to intracellular melanin deposition in organelles called melanosomes. The melanosomes can then be exported from the melanocytes and taken up by adjacent keratinocytes in the skin or by cells proximal to the follicle root sheath.

Compounds useful for the modulation of tyrosinase activity in conjunction with reepithelialization include, without limitation, 5-Bromodeoxyuridine, TPA/insulin, TGF-β1, TNF-α, Agouti signal protein, Hydrogen peroxide, Ceramide, Dihydrolipoic acid/lipoic acid, Sphingosine-l-phosphate, Lysophosphatidic acid, (−)-Epigallocatechin-3-gallate/hinokitiol, Terrein, Piperlonguminine, Sphingosylphosphorylcholine, Glucosamine/tunicamysin, Glutathione, Feldamycin6, N-Butyldeoxynojirimycin, Calcium D-pantetheine-S-sulfonate, Ferritin, Phenylthiourea, Hydroquinone, Azelaic acid, Kojic acid, Dithiothreitol, Arbutin, Magnesium L-ascorbyl-2-phosphate, 2-O-a-D-Glucopyranosyl-L-ascorbic acid, a-Tocopheryl ferulate, 4-Tertiary butylphenol, Bathocuproein disulphonate, Ellagic acid, Aloesin, Bisindolylmaleimide, 4,40-Dihydroxybiphenyl, 4-n-Butylresorcinol, Linoleic acid, 2,20-Dihydroxy-5,50-dipropyl-biphenyl, TPA/phospholipase D2, 25-Hydroxycholesterol, and Phenylthiourea.

Apoptosis Modulators

In yet another embodiment, the invention features administration of compounds that modulate melanocyte apoptosis. Recent studies of interfollicular skin and hair follicle melanocytes have implicated proteins that mediate apoptosis as playing a critical role in the maintenance of normal skin and hair pigmentation. For example, disrupting the Bcl2 gene in melanocytes leads to a significant reduction of melanin production leading to gray hair.

Compounds useful for the modulation of apoptosis in conjunction with reepithelization include, without limitation, Troglitazone, Rolipram, Antineoplaston A10, Genistein, Ukrain, Alvocidib, RO 318220, Dolastatin 10, Diethylnorspermine, Perillyl alcohol, DMXAA, Exisulind, Daunorubicin liposomal, Canfosfamide, Iodine I 131 tositumomab, Colcemid, Cepharanthine, CPENSpm, Betulinic acid, Tangeretin, Oblimersen, Motexafin gadolinium, LDI 200, EL 625, LXR 0152, Irofulven, LXR 0151, Dolastatin 15, Indisulam, E 21R, Bortezomib, Kahalalide F, Usambarensine, Sy 801, LG 100153, Deguelin, Leptofuranin A, Leptofuranin B, Leptofuranin C, Leptofuranin D, Interleukin-4(38-37)-PE38KDELBMLOV, Discodermolide, Alpha-lactalbumin, Anti-Fas IgM monoclonal antibody CH11, Bilobalide, MF 13, Butyrolactone, 2-Methoxyestradiol, t BCEU, Tocopherol succinate, huN901-DM1, PAB 13, PAB 15, PAB 23, CEP 751, Lan 7, MX 33501, MX 28701, MX 901, Beta lapachone, Tilmacoxib, Bcl-2 antagonists (Abbott/Pfizer), apoptosis stimulants (Tripos/MDS/Cell Pathways), INGN 241, DW 2282, Antineoplaston A10, D2A21, BMD 188, DDE 261, WHIP 154, WHIP 131, AP 1903, SR 45023A, Cytotrienin A, Plitidepsin, apoptosis stimulants (Apoptosis Technology), 3-BAABU, AG 17, FE 35A, Elemene, Tipifarnib, GTE TP90, Pralatrexate, Isoharringtonine, GRB2 inhibitors (SUGEN), SBA, CAP 232, LXR 1035, MX 781, Casiopeina II, K 22097, MX 6, OSI 461, WP 401, FE 35B, 3-IAABU, LY 139478, PCK 3145, RTA 401, RO 415253, SNS 595, Idronoxil, CHML, AMG 951, WHIP 232, WHIP 352, WHIP 353, Clofarabine, Diflomotecan, C 857, CP 248, Geranyl tiglate, Arsenic trioxide, Aminoflavone, WHI-D11, EMAP II, Tachpyr, Brostallicin, MS 247, Histamine dihydrochloride, HS 1030, Noscapine, caspase modulators (Molecumetics/Pharmacia), hPRL-G129R, SC-alpha-alpha-delta-9, PC-SPES, MDL 72527, Melarsoprol, CBHA, Polyphenon E, NB 301, Gallic acid, Imexon, indanocine analogues (Salmedix), TS 2, TS 6, apoptosis inducers (Celera Genomics/EpiCept), apoptosis inducers (Nanologix), Obatoclax, 3-BAABE, CAAX 2, apoptosis inducers (Idun/Pfizer Pharmaceuticals), R 440, COBRA-1, Trastuzumab-DM1 immunoconjugate, Recombinant viscumin, colostrinin constituent peptides (ReGen Therapeutics), CATI-1, WP 769, Ispinesib, BAY 361677, TLC 144, SU 9516, HDM2 inhibitors (Johnson & Johnson Pharmaceutical Research and Development, LLC), SPIKET-P, Thymosin beta-4, HA 14-1, DN 1924, Uroguanylin, XIAP, TP 38, BBL 22, 2-ME-D, SiLi D1, Anti-Fas monoclonal antibody RK-8, Anti-Fas monoclonal antibody Jo2, R 125224, Salvicine, E 7389, CGC 11047, PG 49088, Metvan, CS 1008, Anti-Notch-1 monoclonal antibody, Artepillin C, MPC 2130, DJ 927, Anti-CD33 TAP, SDX 101, Mapatumumab, Radiolabelled BLyS, Homoharringtonine, HGS ETR2, PRIMA-1, AEG 35156, ALS 357, apoptosis inducers (EpiCept Corporation), apoptosis inducers (GeminX/Sequoia Sciences), Mitoquinone, AX 200, Pentamidine/chlorpromazine, SJG 136, RIP inhibitors (Apoxis), ST 1926, ESPA 1002, TKI 258, Bcl-2 inhibitors (Kirin Brewery), discodermolide analogues (Cellomics), CSP inhibitors (ArQule), SB 743921, discodermolide analogues (Kosan Bioscience), AS 1411, Imexon (Heidelberg Pharma), Catumaxomab, Rose bengal sodium, CAT 5001, 2-Methoxyestradiol analogues (EntreMed), acylfulvene analogues (MGI Pharma), sapphyrin-derived apoptosis inducers (Pharmacyclics), caspase-3 inhibitors (AstraZeneca), Desmethyldeprenyl (RetinaPharma Technologies), MX 90745 series (EpiCept/Myriad), Ertumaxomab, PBD 2131, RP 101, HDM2 inhibitors (Cyclacel), apoptosis inducers (Aponetics AG), MCC/HA, E2F modulators (ArQule), SDX 101 second generation analogues (Salmedix), polycyclic oxazolidinones (Abbott Laboratories), NV 18, EM 1421, quassinoid therapeutics (Tapestry Pharmaceuticals), labdane diterpene therapeutics (Medexis), ZIO 101, Bcl-2 antagonists (Ricerca), HGS TR2J, PRX 302, curaxins (Cleveland BioLabs), BZL 101, TST 10088, apoptosis stimulants (Genentech), INOC 003, apoptosis stimulants (Novartis), cyanoaziridine derivatives (Amplimed), TRAIL receptor 2 agonist (Affymax), JX 594, apoptosis stimulants (Gentara), RC 8800, SBP 002, XG 102, PM 02734, p53-MDM2 inhibitors (Ascenta Therapeutics), apoptosis stimulants (Ascenta), AT 101, ZIO 102, YM 155, KP 772, MKC 1, TRO 19622, apoptosis stimulants (Bionovo), CBP 501, midkine therapeutics (Cell Signals), Bcl-2 inhibitors (InfmityNovartis), kinesin spindle protein inhibitors (Cytokinetics/GlaxoSmithKline), serratamolide analogues (CRT/University of Barcelona), Urocanic acid (BioCis Pharma), CHER 265, AMG 655, NV 196, Alpha-lactalbumin-oleic acid, apoptosis stimulants (TetraLogic Pharmaceuticals), AFP 464, Rh-Apo2L, TK 54, apoptosis inducers (Advanced Life Sciences), Apomab, JB 991, and BI 2536.

Endothelin Signaling Modulators

In a further embodiment, the invention features administration of compounds that modulate endothelin signaling. Members of the endothelin and endothelin receptor family have been implicated in melanocyte differentiation and proliferation. Specfically, interfering with signaling via the endothelin 3 and the endothelin type B receptor leads to alterations in skin pigmentation.

Compounds useful for the modulation of the endothelin signaling pathway in conjunction with reepithelization include, without limitation, PD 147953, BQ 123, BQ 153, PD 142893, PD 145065, PD 151242, RO 462005, U 88999E, 50 235, SPI 1620, SB 209670, TAK 044, Bosentan, BQ 610, Enrasentan, BMS 182874, PD 156252, CGS 27830, L 749329, L 744453, BQ 485, PD 156707, PD 155080, CGS 26303, L 746072, IRL 2500, PD 159433, L 754142, A 127722, BQ 518, WS 75624B, EMD 94246, PD 159020, Sitaxsentan, TAK 225, RES 7011, PD 161721, RPR 111844, Darusentan, BMS 193884, SCH 54470, LU 127043, A 182086, A 206377, Atrasentan, PD 166557, PD 164997, PD 163070, L 749805, PD 166114, S 17162, SB 215355, ZD 1611, SB 234551, S 0139, CGS 31447, ZD 2574, ZD 4054, RO 485695, A 192621, SB 247083, A 200379, A 183491, RO 611790, J 104132, Daglutril, TA 0201, Fandosentan, ABT 546, LU 302872, TBC 11241, TBC 11192, PD 166309, CGS 26582, Tezosentan, LU 224332, ATZ 1993, FR 901533, YM 598, TMC 66, PD 164800, BQ 788, TBC 3711, TBC 3214, PABSA, RPR 118031A, K 8794, K 8768, Ambrisentan, Avosentan, SM 19712, J 112534, A 292438, Edonentan, J 105859, BSF 302146, YM 62899, A 306552, Clazosentan, and endothelin B receptor agonists (Spectrum Pharmaceuticals).

Nuclear Receptor Pathways (e.g., Retinoic Acid and Vitamin D)

In another embodiment, the invention features administration of compounds that modulate nuclear receptor pathways, such as the retinoic acid or the vitamin D signaling pathways. These pathways have been linked to initiating the differentiation of melanocyte precursors into melanoblasts and later stage melanasomes during embryogenesis. Modulating these pathways in conjunction with reepithelization could lead to an effective treatment for skin pigment disorders.

Retinoids signal via two classes of nuclear receptors: retinoic acid receptor (RAR) and retinoic X receptor (RXR). Vitamin D and its primary metabolite, 1,25(OH)₂D₃, signal through the vitamin D receptor which is a member of the steroid receptor superfamily.

Compounds useful for the modulation of the retinoic acid and vitamin D signaling pathway in conjunction with reepithelization include, without limitation, trans-retinoic acid, N-retinoyl-D-glucosamine, seletinoid G, Fenretinide, Liarozole, Tazarotene, AM 580, Bexarotene, Alitretinoin, AR 623, AGN 191701, SR 11237, CGP 52608, LG 100153, LGD 1550, LG 100567, AGN 193835, AGN 193836, MX 33501, MX 28701, MX 901, MDI 403, LGD 1324, AGN 194310, CD 437, UAB 8, CD 1599, TAC 101, SR 11383, LGD 1268, 4-Oxoretinol, ER 35794, BMS 185411, RO 415253, ER 38925, ER 65250, R 116010, BMS 292974, UAB 30, VN/14-1RA, BMS 297208, LG 101506, Tretinoin, L 007, Isotretinoin, PLT 99511, AGN 195183, AGN 194204, R 667, retinoid X receptor alpha agonists, retinoic acid receptor gamma agonists (Locus), BMS 189453, retinoic acid metabolising enzyme blockers (Bioenvision), LXS/4-HPR, Seletinoid G, Rambazole, fenretinide, Carbenoxolone, Maxacalcitol, Seocalcitol, Falecalcitriol, Doxercalciferol, RO 245531, Calcipotriol, MC 1288, RO 237553, ST 232, CB 1267, 1-alpha-Hydroxyvitamin D5, Tacalcitol, Inecalcitol, Paricalcitol, RO 259716, Calcitriol, RO 256760, RO 270574, Gemini, Atocalcitol, RO 269228, 1-alpha-Hydroxy-24-epi-vitamin D5, BAL 2299, RO 262198, DN 101, BXL 353, BXL 490, vitamin D3 analogues (BioXell/ProStrakan), RC 8800, Ostabolin, vitamin D3 analogues (Schering AG), vitamin D analogues (Aphios/Boston University), vitamin D3 derivatives (Astellas Pharma), Calcithiazol, and BXL 746.

TGFβ-SMAD, Bone Morphogenetic Protein, and Stem Cell Factor Signaling Pathways

In one embodiment, the invention features administration of compounds that modulate the TGFβ-SMAD signaling pathway and the bone morphogenetic protein (BMP) pathways. Members of both the BMP and TGFβ signaling pathways have been implicated in early melanocyte precursor proliferation and differentiation during embryogenesis.

BMPs are secreted proteins that broadly regulate of cell proliferation, differentiation, and apoptosis by signaling through BMP receptors. Biochemical analysis of BMP-mediated signaling suggest that BMPs interact with other protein families including Wnt, Shh, TGF-β, EGF, FGF, Notch, and others.

During development, BMP-6 is expressed primarily in the suprabasal layers of the epidermis, and BMP-7 is found primarily basal layer of the epidermis. Expresison of BMP-2 and BMP-4 is restricted primarily to the developing hair follicle. BMP receptors-IA and BMP receptors-4B are restricted to suprabasal keratinocytes. Smad1, Smad5, Smad6, and Smad7, downstream signaling molecules in the BMP pathway, are also expressed in the developing epidermis.

During embryonic hair follicle induction, BMP-2 and BMPR-IA are found in the hair placode while BMP-4 and noggin, an endogenous inhibitor of BMP signaling, are observed in the mesenchymal cell layer below the thickening epidermis. BMP-2 signaling is also implicated in dermal remodeling, potentially via an interaction with the matrix metalloproteinase (MMP) family of extracellular matrix (ECM)-degrading enzymes.

Modulation of molecular signaling in embryonic and adult skin are commonly mediated through the TGFβ-SMAD pathway. One of the major downstream pathways is the synthesis of collagen 1, the primary collagen in adult dermis. Sphingosine 1-phosphaste and asiaticoside are naturally occurring molecules that can enhance collagen production via TGFβ1/2-SMAD signaling (Lee J. et al. (2006) Planta Med 72:324-28 and Cuidan X. et al. (2004) JBC 279:35255-62). TGF-β3 is also expressed in the skin and plays a role in the development and regulation of numerous processes including pigmentation.

In one embodiment, the invention features administration of compounds that modulate stem cell factor (SCF) signaling. In addition to being a molecular marker for melanoblasts, the SCF/KIT signaling pathway is known to play a critical role in melanocyte development. Interfering with the SCF singaling pathway leads to changes in melanoblast and melanocyte vialability through selective activation of apopotsis pathways.

Compounds useful for the modulation of the TGFβ-SMAD, BMP, and/or SCF pathways in conjunction with reepithelialization include, without limitation: Eptotermin alfa, Noggin, bone morphogenetic protein activators (Curis/Ortho Biotech), Transforming growth factor-beta-3, Transforming growth factor-beta-1, Transforming growth factor-alpha, Cetermin, Tamoxifen methiodide, Decorin, Kahalalide F, Anti-TGF-beta monoclonal antibody 2G7, ADMP 1, Lerdelimumab, Metelimumab, TGF-beta antagonists (GLYCODesign), A 161906, LF 984, Tetrathiomolybdate, Tranilast, GC 1008, SEK 1005, TGF-beta antagonists (Scios), SR2F, Stamulumab, NeuGene antisense compounds (AVI BioPharma), TJN 598, TGF-beta RI kinase inhibitors (Scios), TGF-beta oligonucleotide nanoparticles (NanoDel), TGF-beta type I receptor inhibitors (In2Gen), TG-C, and Mannose 6 phosphate.

Cytokine Signaling

In yet another embodiment, the invention features administration of compounds that modulate cytokine and growth factor signaling. Pro-inflammatory cytokines, including interleukin-1 (IL-1), interleukin-8 (IL-8), TNF-α, IL-6, and interferon γ and interferon α, among others, have been linked to inflammatory-induced changes in pigmentation. Modulation of the cytokines are thought to significantly contribute to changes in pigmentation by altering the expression and/or activity of MCR-1. The expression of the genes that code for the proinflammatory cytokines is upregulated via nuclear factor kappaB (NF-kappaB) and AP-1, well known proinflammatory transcription factors. Therefore, stimuli that induce up-regulation of the NF-kappaB pathway contribute to the alteration of the levels of proinflammatory cytokines and therefore pigmentation disorders.

Compounds useful for the modulation of cytokine and growth factor in conjunction with reepithelialization include, without limitation, Imiquimod/Avara, IL-1alpha, parthenolide, magnolia extract, magnolol, Prasterone, Iguratimod, Suplatast tosilate, Bindarit, Liarozole, UK 122802, ONO 4007, Stiripentol, DUP 983, DUP 630, DMXAA, ICZ, FPP 33, PP 33, Mesoporphyrin, Semapimod, A 802715, Pirfenidone, Sho-seiryu-to, FR 167653, Pentoxifylline, Iboctadekin, Pimecrolimus, Temsirolimus, REP 689, R 116010, Tadekinig alfa, Prasterone, PB 007, anti-interleukin-18 monoclonal antibodies (CAT), ISIS 104838, Delmitide, P450RAI inhibitors (Cytochroma), ZNC 2381, R 115866, CLX 0921, Thymosin beta-4, M 50367, JTE 607, Licochalcone A, vitamin D signal amplifiers (Cytochroma), TS 011, CF 101, Prasterone phosphocholine, Y 39041, RDP 58 analogues (Genzyme/Synt:em), NPI 1302a-3, AVI 4557, Susalimod, p38 MAP kinase inhibitors (Uriach/Organon), MT 201, interleukin-4/5 secretion inhibitors (Fournier/Zambon), LMP 160, LMP 420, PLR 14, AD-GL0001, CLX 090717, CLX 090502, CRX 102, Ciclosporin, ABT 325, IMS, K 832, CC 10004, Interleukin 6 inhibitor (Y's Therapeutics), YSTH2, CR1 (Nuada), CRX 119, CRX 139, Golimumab, Rambazole, cytokine receptor antagonists (Trillium Therapeutics), high mobility group box chromosomal protein-1 inhibitors (Nautilus/Creabilis), CYT 007, TNFQb, QR 440, CTA 018, K 412, AN 0128, CRX 170, CRx 140, CRX 150, RC 8800, tumour necrosis factor gene therapy (Onc Bio), Recombinant IL-18 binding protein, HMPL 004, tpl2 kinase inhibitors, SPC 839, RTA 401, MPC 7869, INDRA compounds (Active Biotech), APC 0576, NF-kappa-B Decoy oligonucleotide (Anesiva), BG 12, NF-kappa B/IKK2 inhibitors (Uriach), Antisense oligonucleotide NF-KappaB-p65 (Serono/InDex Pharmaceuticals), I-kappa B kinase inhibitors (Millennium Pharmaceuticals), NeuGene antisense compounds (AVI BioPharma), SIM 916, liposomal calagualine (Plantacor), NF-kappa B inhibitors (Serenex), SIM 688, NF-kappa B inhibitors (Scottish Biomedical), NFkappaB pathway inhibitors (4SC), Synthetic triterpenoids (Reata Pharmaceuticals), RTA 402, Mecasermin, rinfabate, Insulin-like growth factor-II, CEP 903, INX 4437, 486-STOP, carbohydrate-based anti-inflammatories (Praxis/Fairchild), MZ 471, and MZ 5156.

### Compounds to be Administered During Reepithelialization to Treat Acne

Following induction of reepithelialization, therapeutic compounds may be applied to the skin according to the methods of the invention. Such therapeutic compounds are, for example, compounds known to treat acne and compounds known to modulate signaling pathways associated with acne (e.g., the pathways described below).

Androgen Pathways

In one embodiment, the invention features administration of compounds that modulate androgen signaling in the skin. Increased androgen signaling in the skin has been definitively linked to increased hair follicle size and increased sebaceous gland growth and differentiation that are critical for the onset of acne. In fact, acne development clearly parallels the increase in androgen levels through puberty and wanes in the later teenage years as androgen levels plateau. Also, people with limited or a complete lack of androgen signaling do not develop acne.

Compounds and enzymes useful for treating acne by reducing androgen levels in the skin in conjunction with reepithelialization include, without limitation Bicalutamide, Zanoterone, Osaterone, Cioteronel, Nilutamide, WB 2838, PSK 3841, LG 2293, Louisianin A, SR 4980, SNA 4606, Abarelix, ZD 3980, LGD 1331, Elaiophylin, Efomycin G, L 10, L 39, L 35, L 37, L 2, I 41, VN 851, PH 45, Cyproterone acetate (Barr Laboratories), androgen receptor antagonists (Karo Bio), selective androgen receptor antagonists (Biogen Idec), androgen receptor antagonists (Praecis), selective androgen receptor modulators (GTx), androgen receptor antagonists (Bristol-Myers Squibb), androgen receptor antagonists (Astellas Pharma), Ostarine, and androgen receptor antagonists (Medivation).

Retinoic Acid Pathway

In another embodiment, the invention features administration of compounds that modulate the retinoic acid signaling pathway. This pathway has been linked to pilosebaceous gland morphogenesis, including sebaceous gland formation, likely through the mediation of epidermal-mesenchymal interactions in the embryonic follicle. Retinoids also have a profound effect on the activity of sebaceous glands: trace amounts promote sebocyte growth and differentiation, while larger doses lead to sebocyte atrophy and decrease sebum production. Retinoids signal via two classes of nuclear receptors: retinoic acid receptor (RAR) and retinoic X receptor (RXR).

Compounds useful for the modulation of the retinoic acid signaling pathway in conjunction with reepithelialization include, without limitation, trans-retinoic acid, N-retinoyl-D-glucosamine, seletinoid G, Fenretinide, Liarozole, Tazarotene, AM 580, Bexarotene, Alitretinoin, AR 623, AGN 191701, SR 11237, CGP 52608, LG 100153, LGD 1550, LG 100567, AGN 193835, AGN 193836, MX 33501, MX 28701, MX 901, MDI 403, LGD 1324, AGN 194310, CD 437, UAB 8, CD 1599, TAC 101, SR 11383, LGD 1268, 4-Oxoretinol, ER 35794, BMS 185411, RO 415253, ER 38925, ER 65250, R 116010, BMS 292974, UAB 30, VN/14-1RA, BMS 297208, LG 101506, Tretinoin, L 007, Isotretinoin, PLT 99511, AGN 195183, AGN 194204, R 667, retinoid X receptor alpha agonists, retinoic acid receptor gamma agonists (Locus), BMS 189453, retinoic acid metabolising enzyme blockers (Bioenvision), LXS/4-HPR, Seletinoid G, Rambazole, fenretinide, and Carbenoxolone.

Peroxisome Proliferator-Activated Response Receptors

In another embodiment, the invention features administration of compounds that modulate peroxisome proliferator-activated response receptor (PPAR) family. PPARs are nuclear hormone, ligand-induced transcription factors that generally act as cellular sensors of polyunsaturated fatty acids and other fatty acid derivatives. PPARα and PPARβ are both expressed at different times and areas of embryonic and adult skin, while PPARδ has been linked to sebaceous gland lipid production. PPARα is expressed in the adult skin after injury and plays an important role in mediating the initial inflammatory-mediated healing response. PPARα and PPARβ are both constitutively expressed in the hair follicle where they are thought to play an active role in mediating the hair follicle cycle. In addition to its role in hair follicle development, PPARβ also plays a critical role in mediating skin repair in response to injury. Finally, PPAR expression has been tied to lipid production which may have specific relevance for sebum production in sebaceous follicles. Therefore, selective inhibitors of the PPAR subtypes could lead to novel acne treatments.

Compounds useful for the modulation of the PPAR signaling pathway in conjunction with reepithelialization include, without limitation, Troglitazone, Pioglitazone, Englitazone, AY 31637, Darglitazone, Rosiglitazone, Ciglitazone, AD 5075, Bexarotene, Netoglitazone, BM 131246, BM 501050, Farglitazar, Balaglitazone, Reglitazar, GW 2570, GW 409890,Tesaglitazar, MK 0767, PD 72953, Ragaglitazar, GW 409544, Rivoglitazone, GW 1929, GW 9578, GW 0072, SB 219994, LG 101506, Metaglidasen, CLX 0921, LR 90, LY 510929, GW 501516, Naveglitazar, NC 2100, PPAR gamma antagonists (Bayer/GSK), LF 200337, GW 5393, PPAR alpha/gamma agonists (Eli Lilly), Muraglitazar, ARH 049020, MBX 2044, KT 6207, GW 7282, PPAR alpha/gamma agonists (Bayer), GW 590735, BAY 549801, L 764406, CLX 0940, NS 220, PPAR gamma agonists (Vita), Fenofibrate, 677954, LY 518674, AMG 131, KRP 101, PPAR agonists (Merck & Co), DRF 4832, ONO 5129, Fenofibrate/metformin, Oxeglitazar, PPAR agonists (GlaxoSmithKline), TY 51501, AA 10090, peroxisome proliferator-activated receptor agonists (Karo Bio), PPAR modulators (Fournier Pharma), AZD 6610, 641597, PPAR delta agonists (Nippon Chemiphar/Pfizer), PPAR pan agonists (Plexxikon), DRF 10945, AVE 0847, PPAR-gamma agonists (Daiichi Sankyo), peroxisome proliferation activated receptor beta modulators (7TM Pharma), Peliglitazar, PPAR alpha agonists (CrystalGenomics), PPAR alpha/gamma agonists (MaxoCore Pharmaceuticals), AVE 8134, PPAR alpha agonists (MaxoCore Pharmaceuticals), peroxisome proliferator-activated receptor agonists (Novo Nordisk), PPAR delta agonists (Eli Lilly), E 3030, PPAR agonists (Metabolex), DRL 11605, LBM 642, peroxisome proliferator-activated receptor alpha/gamma agonists (Sanofi-Aventis), PLX 204, peroxisome proliferator-activated receptor modulators (AbGenomics), PPAR delta agonists (Nippon Chemiphar/Cerenis), Fenofibrate/simvastatin, 625019, CS 7017, CKD 501, AVE 5376, PPAR delta agonists (sanofi-aventis), Ezetimibe/fenofibrate, RWJ 800025, Fenofibrate/rosuvastatin calcium, AB 335/rosuvastatin calcium, Fenofibrate/rosuvastatin, Pioglitazone/TAK 536, CDT-fenofibrate, PPAR agonists (Bayer), peroxisome proliferator-activated receptor agonists (Eli Lilly), KD 3010, GFT 505, LG 101506, Metaglidasen, LY 510929, Naveglitazar, NC 2100, PPAR gamma antagonists (Bayer/GSK), MBX 2044, BAY 549801, PPAR modulators (Fournier Pharma), peroxisome proliferation activated receptor beta modulators (7TM Pharma), and peroxisome proliferator-activated receptor modulators (AbGenomics).

Estrogen Signaling

In yet another embodiment, the invention features administration of compounds that modulate estrogen signaling. Estrogens are known to effect numerous skin-related conditions, including acne onset and severity. Women have a steady deterioration in their skin architecture after menopause which can be reversed by hormone replacement therapy (HRT). Topical application of 17β-estradiol has been shown to mimic these effects without the peripheral side effects of hormone replacement therapy (HRT) (Verdier-Sevrain et al. (2006) Exp Dermatol 15:83-94 and Son E D et al. (2005) JID 124: 1149-61).

Compounds useful for the modulation of estrogen signaling pathways in conjunction with reepithelialization include, without limitation, 17β-estradiol, estriol, estrone, conjugated estrogens (e.g., Premarin, PremPro), diethylstilbestrol selective ER modulators (SERMS) (e.g., tamoxifen, raloxifene, toremifene, clomifene, bazedoxifene, lasofoxifene, and ormeloxifene), Fulvestrant, ICI 164384, Zindoxifene, Panomifene, CB 7386, RU 39411, LY 133314, RU 58668, ZK 119010, EMATE, Prolame, WS 7528, RU 16117, Yuehchukene, 3-Methyl-3-hydroxy-chalcone, Tesmilifene, RU 45144, CDRI 85287, Tamoxifen methiodide, Estradiol/trimegestone, ICZ, EM 219, Ethinylestradiol/gestodene monophasic, Ethinylestradiol/drospirenone, Complex K, ZK 115194, Ethinylestradiol/dienogest, J 893, BE 25327, Estradiol valerate/dienogest, TS 17, Abarelix, Estradiol/norethisterone, Estradiol/levonorgestrel, Ethinylestradiol/gestodene-triphasic, Centchroman, TS 33, EM 800, Estradiol/nomegestrol, SR 90067, OSW-1, K 7, Anordrin, Ospemifene, Alpha-Fetoprotein, Estradiol/testosterone, IP 1162, IP 1163, IP 1164, J 995, estrogen receptor-alpha antagonists (Sumitomo), Estradiol/norethisterone, Ethinylestradiol/desogestrel, Estradiol cipionate/medroxyprogesterone, Ethinylestradiol/levonorgestrel, Ethinylestradiollnorethisterone, Esterified estrogens, Ethinylestradiol/levonorgestrel, Ethinylestradiol/norethisterone, Ethinylestradiol/chlormadinone, Conjugated estrogens, Estradiol/dydrogesterone, Trilostane, Ethinylestradiol/etonogestrel, P 081, Ethinylestradiol/norgestimate, EMM 210525, Estradiol acetate vaginal, Estradiol/progestogen, SM 16896, Acolbifene, Estradiol valerate/medroxyprogesterone, Ethinylestradiol/gestodene, NNC 450095, SDN 289, TZN 13, BAY 509062, MCC 565, NV 50, Estradiol/nomegestrol, EMM 310525, PSK 3987, S 401G, pure estrogen receptor antagonists (ProStrakan), E2CDS, J 811, J 861, Afimoxifene, Enclomifene, Estradiol/dienogest, BN 83495, SIM 916, ERB 196, Tamoxifen, SIM 688, AP 1081, estrogen-related receptor alpha modulators (Phenex Pharmaceuticals), ORG 43228, and 8-Prenylnaringenin.

Cytokine and Growth Factor Signaling

In yet another embodiment, the invention features administration of compounds that modulate cytokine and growth factor signaling. Pro-inflammatory cytokines, including interleukin-1 (IL-1), interleukin-8 (IL-8), TNF-α, IL-6, and interferon γ and α, among others, are up-regulated in response to inflammatory acne. Modulation of the cytokines are thought to significantly contribute to the formation of acne scar lesions commonly associated with prolonged inflammatory acne. The expression of the genes that code for the proinflammatory cytokines is upregulated in response to nuclear factor kappaB (NF-kappaB) and AP-1, well known proinflammatory transcription factors. Therefore, stimuli that induce up-regulation of the NF-kappaB pathway contribute to the alteration of the levels of proinflammatory cytokines and therefore inflammatory acne.

Numerous growth factors, including but not limited to members of the fibroblast growth factor (FGF) family (including keratinocytes growth factor), epithelial growth factor (EGF), insulin-like growth factor (IGF), hepatocyte growth factor, growth hormone (GH), insulin, and platelet-derived growth factor (PDGF), have also been implicated in acne. For example, GH, IGF, FGF and insulin have all been implicated in sebaceous gland morphogenesis as well as sebum production in the adult.

Compounds useful for the modulation of cytokine and growth factor signaling in conjunction with reepithelialization include, without limitation, Imiquimod/Avara, IL-1alpha, parthenolide, magnolia extract, magnolol, Prasterone, Iguratimod, Suplatast tosilate, Bindarit, Liarozole, UK 122802, ONO 4007, Stiripentol, DUP 983, DUP 630, DMXAA, ICZ, FPP 33, PP 33, Mesoporphyrin, Semapimod, A 802715, Pirfenidone, Sho-seiryu-to, FR 167653, Pentoxifylline, Iboctadekin, Pimecrolimus, Temsirolimus, HEP 689, R 116010, Tadekinig alfa, Prasterone, PB 007, anti-interleukin-18 monoclonal antibodies (CAT), ISIS 104838, Delmitide, P450RAI inhibitors (Cytochroma), ZNC 2381, R 115866, CLX 0921, Thymosin beta-4, M 50367, JTE 607, Licochalcone A, vitamin D signal amplifiers (Cytochroma), TS 011, CF 101, Prasterone phosphocholine, Y 39041, RDP 58 analogues (Genzyme/Synt:em), NPI 1302a-3, AVI 4557, Susalimod, p38 MAP kinase inhibitors (Uriach/Organon), MT 201, interleukin-4/5 secretion inhibitors (Fournier/Zambon), LMP 160, LMP 420, PLR 14, AD-GL0001, CLX 090717, CLX 090502, CRX 102, Ciclosporin, ABT 325, IMS, K 832, CC 10004, Interleukin 6 inhibitor (Y's Therapeutics), YSTH2, CR1 (Nuada), CRX 119, CRX 139, Golimumab, Rambazole, cytokine receptor antagonists (Trillium Therapeutics), high mobility group box chromosomal protein-1 inhibitors (Nautilus/Creabilis), CYT 007, TNFQb, QR 440, CTA 018, K 412, AN 0128, CRX 170, CRx 140, CRX 150, RC 8800, tumour necrosis factor gene therapy (Onc Bio), Recombinant IL-18 binding protein, HMPL 004, tpl2 kinase inhibitors, SPC 839, RTA 401, MPC 7869, INDRA compounds (Active Biotech), APC 0576, NF-kappa-B Decoy oligonucleotide (Anesiva), BG 12, NF-kappa B/IKK2 inhibitors (Uriach), Antisense oligonucleotide NF-KappaB-p65 (Serono/InDex Pharmaceuticals), I-kappa B kinase inhibitors (Millennium Pharmaceuticals), NeuGene antisense compounds (AVI BioPharma), SIM 916, liposomal calagualine (Plantacor), NF-kappa B inhibitors (Serenex), SIM 688, NF-kappa B inhibitors (Scottish Biomedical), NFkappaB pathway inhibitors (4SC), Synthetic triterpenoids (Reata Pharmaceuticals), RTA 402, Mecasermin, rinfabate, Insulin-like growth factor-II, CEP 903, INX 4437, 486-STOP, carbohydrate-based anti-inflammatories (Praxis/Fairchild), MZ 471, MZ 5156, IGF-1 receptor inhibitors (Telik), HF 0299, EN 122002, IGF-related antagonists (Novo Nordisk/DGI BioTechnologies), NBI 31772, Pasireotide, Rinfabate, OGX 225, mono-specific IGFBP inhibitors (OncoGeneX), IMC A12, IGF-1R kinase inhibitors (Novartis), anti-IGF-1R antibody (Schering Plough), CP 751871, ATL 1101, anti-IGF-1 monoclonal antibody (Pierre Fabre Medicament/Merck), INSM 18, AVE 1642, insulin-like growth factor-1 receptor antagonists (Insmed), insulin-like growth factor therapeutics (Chlorogen), AMG 479, insulin-like growth factor binding protein 1 (Amgen), Troglitazone, Pioglitazone, Glimepiride, Englitazone, Insulinotropin, CP 95253, Talibegron, Darglitazone, Rosiglitazone, RX 871024, U 10483, Glucagon-like peptide-17-36 amide, Ciglitazone, S 15261, AD 5075, Bexarotene, Formycin A, BM 130913, BM 131074, BM 170505, BM 131196, BM 131188, BM 131180, Netoglitazone, BM 131246, BM 501050, BM 131215, TLK 16998, V 411, Glisentide, AZM 134 (Alizyme), Farglitazar, INS 1, Balaglitazone, Reglitazar, LY 315902, SDZ PGU 693, GW 2570, Glucagon-like peptide-1 (Amylin), Glucagon-like peptide-1 (Watson), YM 440, TS 971, DRF 2189, MK 0767, DN 108, HQL 975, Galparan, LGD 1268, T 174, JTT 608, K 111, Ragaglitazar, KP 102, Dexlipotam, YM 268, Rivoglitazone, Tifenazoxide, Metformin, SB 219994, JTP 20993,R 102380, insulin sensitisers (Incyte Corporation), FK 614, CLX 0900, CLX 0901, CLX 0100, CLX 0101, ALT 4037, insulin sensitisers (Roche), BM 152054, Glibenclamide/metformin, DRF 554158, Metaglidasen, CLX 0921, DRF-NPPC, LP 100, LY 307161, BL 11282, BL 11778, NN 570014, NIP 223, NIP 221, MBX 2044, MBX 675, insulin receptor activators (Telik), Y 39677, Edaglitazone, ONO 5816, TLK 17411, EN 122001, EN 122004, BIM 23268, EML 4156, Imiglitazar, KF 72926, ST 1863, insulin mimetics (Merck & Co), BLX 2001, insulin sensitisers (Wellstat Therapeutics), Fenofibrate/metformin (Fournier Pharma), Glipizide/metformin (Bristol-Myers Squibb), Rosiglitazone/metformin, Sipoglitazar, Metformin/sulfonylurea (DepoMed), SMP 862, oral insulin/insulin sensitiser (Diabetology), Pioglitazone/metformin, Pioglitazone/glimepiride, Rosiglitazone/glimepiride, 625019, MK 0431, CKD 501, Pioglitazone/TAK 536, Lanreotide, Seglitide, Vapreotide, Sermorelin, BIM 28011, Somatropin, L 692429, Examorelin, Pralmorelin, G120R, JO15X, Octreotide, NSAC compounds (Sapphire Therapeutics), SR 29001, Ibutamoren, TH 9506, Palifermin, L 163833, L 163689, Teduglutide, Mutant somatropin (JCR Pharmaceutical), NNC 260161, Tabimorelin, Somatorelin, Pegvisomant, Tesamorelin, L 163540, NNC 260722, NNC 260762, L 165034, L 168721, AOD 9604, somatropin mimetics (Celera Genomics/Pfizer), Ipamorelin, L 739943, Capromorelin, G120K PEG, L 165666, LY 438434, LY 444711, LY 426410, EP 51216, L 163255, somatostatin analogues (Ardana), CP 45959901, EP 51389, SM 130686, BIM 23244, GHRP-1, S 37435, Albumin/somatropin, CP 464709, L 166446, Velafermin, CJC 1295, Pasireotide, EP 1572, PEG-GHRF, ghrelin antagonists (AEterna Zentaris), ATL 1103, TZP 101, MTC-Octreotide, RC 1291, Sermorelin (LAB International), GHRH antagonists (AEterna Zentaris), (growth hormone-related compounds (Neuren), long-acting growth hormone (Bolder BioTechnology), growth hormone secretagogues (Elixir Pharmaceuticals), CAM 2029, CYT 009, GhrQb, GTP 200, SUN 11031, long-acting growth hormone receptor antagonist (DiAthegen), PHA 794428, Epidermal growth factor, RG 13022, Leflunomide, RG 83852, EMD 55900, Reveromycin A, Heparin-EGF-like factor, OLX 103, Anti-EGFR monoclonal antibody 528, Cetuximab DAPH 1, Epidermal growth factor fusion toxin, PD 153035, Anti-EGFR monoclonal antibody-DM1 conjugate, MDX 447, Monoclonal antibody MINTS, Matuzumab, CGP 59326, SU 5271, Gefitinib, CGP 62706, Monoclonal antibody 108, Monoclonal antibody B4G7-gelonin conjugate, Panitumumab, Erlotinib, Anti-EGFR sheep monoclonal antibody, epidermal growth factor receptor tyrosine kinase inhibitors (AstraZeneca), CGP 74321, CGP 76627, PD 169540, PD 168393, PD 160678, RG 8803, PD 169414, DAB 720, CP 292597, scFv(14E1)-ETA, small molecule HER-2 inhibitors (Cengent Therapeutics), BIBX 1382, Canertinib, PD 158780, PD 165557, PD 166075, CL 387785, Nimotuzumab, SU 5502, SU 5501, SU 5503, SU 5504, SU 5228, Thiazinotrienomycin B, Anti-EGFR catalytic antibody (Abgenix), Vandetanib, Sporostatin, EKI 785, PM 166, Anti-EGFR monoclonal antibody-Y-90/Re-188, Epidermal growth factor-genistein, CRM 197, Anti-EGFR monoclonal antibody-Tc-99, Pelitinib, Lapatinib, PX 1041, PX 1031, Zalutumumab, Anti-EGFR monoclonal antibody KSB 107, DWP 401, Pazopanib, SC 100, EGFR/ErbB2 inhibitors (Array BioPharma), MDX 214, ALT 110, IMC 11F8, EGFRvIII antibody drug conjugates (Amgen), Anti-EGFR monoclonal antibody 806, XL 647, BMS 599626, INCB 7839, EGFR/HER2 tyrosine kinase inhibitors (Mitsubishi Pharma), epidermal growth factor kinase inhibitors (ImClone Systems), ARRY 334543, BIBW 2992, and anti-EGFR proteins (Med Discovery).

Toll-Like Receptor Signaling

In yet another embodiment, the invention features administration of compounds that modulate toll-like receptor (TLR) signaling. The TLR family of cell surface receptors includes ten known family members in humans generally involved in pathogen recognition and innate immune system stimulation. Several studies identified differential TLR 1, 2, 4, 5, and 9 expression in human keratinocytes at different levels of the skin. Alteration in normal TLR expression has been associated with a variety of human skin diseases and disorders, including acne, leprosy, and psoriasis. In particular, high levels of TLR2 expression in macrophages associated with pilosebaceous glands in acne lesions indicates a roll for this TLR subtype in acne.

Compounds useful for the modulation of TLR signaling in conjunction with reepithelialization include, without limitation, OM 174, CpG 7909, Eritoran, Isatoribine, toll-like receptor 9 agonists (Idera Pharmaceuticals), IMO 2055, CpG 10101, toll-like receptor 4 modulators (GlaxoSmithKline), toll-like receptor 7/8 agonists (Idera Pharmaceuticals), TLR9 agonists (Coley/sanofi-aventis), CRX 675, TLR9 antagonists (Coley), next-generation toll-like receptor 9 agonists (Coley/Pfizer), Sotirimod, toll-like receptor 3 agonists (Innate Pharma), and toll-like receptor 9 agonists (AstraZeneca/Dynavax).

Neurotrophin and Neuroendocine Signaling

In yet another embodiment, the invention features administration of compounds that modulate neurotrophin (NT) signaling in the skin. The neurotrophin family is composed of nerve growth factor (NGF), brain-derived growth factor (BDNF), neurotrophin-3 (NT-3) and neurotrophin-4 (NT-4). High affinity NT receptors belong to the tyrosine kinase family and include TrkA, TrkB, and TrkC. The NT's also interact with p75NTR, albeit with a lower affinity. NGF, NT-3, and BDNF are expressed primarily by fibroblasts, although expression has also been noted in cutaneous nerve fibers and myocytes in the arrector pili and panniculus carnosus muscles. Proliferating human keratinocytes produce and secrete NGF. TrkA and TrkB are primarily expressed on epidermal keratinocytes whereas TrkC is found on cutaneous nerve cells and in the hair follicle.

The onset of NT expression is observed early in murine embryonic development in the skin epithelium and mesenchyme, and correlates with epidermal K5 and K14 expression. Maximal embryonic expression correlates with hair follicle induction in murine dorsal skin. NT's also play a critical role in melanoblast and melanocyte migration, viability, and differentiation during embryogenesis.

Substance P expression on nerve fibers proximal to sebaceous glands in skin from patients with acne suggested a possible role for this pathway in acne pathogenesis. Corticotropin-releasing hormone (CRH) and the corresponding CRH receptors are also present in human sebaceous glands where they are thought to play a role in regulating sebocyte activity in response to physical insult. In addition, the sebaceous gland is a target for a-melanocyte stimulating hormone (α-MSH) the effects of which are mediated through the melanocortin-1 receptor.

Compounds useful for the modulation of neurotrophin and neuroendocine signaling in conjunction with reepithelialization include, without limitation, phorbol 12-tetra decanoate 13 acetate, FK 224, RP 67580, CP 99994, GR 73632, Cizolirtine, Peptide G, Peptide D, L 732138, DAB389, substance P, RP 73613, RPR 111905, Aprepitant, Ezlopitant, AA 501, AV 608, ESP7, E 6006, L 759274, MIF 1, CUV 1647, HP 228, Nemifitide, PT 14, RO 273225, melanocortin-4 receptor antagonists (Gene Logic), melanocortin-4 receptor agonists (Pharmacopeia, Melacure Therapeutics, Merck, Palatin Technologies, Novo Nordisk, Amgen, Ipsen, LG Life Sciences, Eli Lilly, Amura), melanocortin-4 receptor modulators (Neurocrine Biosciences), Bremelanotide, melanocortin-4 receptor agonists (LION bioscience/Novartis), TRG 2411, ZYC 200, CZEN 002, melanocyte stimulating hormone analogues (Zengen), melanocortin receptor antagonists (Taisho), melanocortin-4 receptor antagonists (Santhera Pharmaceuticals), CZEN 003, melanocortin-4 receptor modulators (TransTech Pharma), melanocortin receptor antagonists (Palatin Technologies), AP 214, RO 0282425, melanocortin receptor agonists (AnaMar Medical), Corticorelin, CP 154526, CRH 9 41, SC 241, Corticotropin releasing factor antagonist (Pfizer), NBI 30775, SJ 948, DMP 695, SP 904, corticotropin releasing factor receptor antagonists (Taisho), PD 171729, NGD 981, NBI 30545, NBI 31199, NBI 31200, DMP 696, NBI 27155, Urocortin, SV 030, IL 488, GSK 876008, NBI 34041, SSR 125543, NGD 982, corticotrophin releasing factor antagonists (Neurogen), AVE 4579, AAG 561, Corticotropin-releasing factor 1 antagonist (Bristol-Myers Squibb), corticotropin releasing factor R1 and R2 receptor antagonists (Neurocrine/GlaxoSmithKline), ONO 2333, TS 041, and corticotropin releasing factor R1 receptor antagonists (Sanofi-Aventis).

### Compounds to be Administered During Reepithelialization to Alleviate or Prevent Scar Formation

Following induction of reepithelialization, therapeutic compounds may be applied to the skin according to the methods of the invention. Such therapeutic compounds are, for example, compounds known to alleviate or prevent scar formation and compounds known to modulate signaling pathways associated with scar formation (e.g., the pathways described below).

TGF-β Signaling Pathways

The transforming growth factor (TGF) family of proteins has pro-fibrotic functions in promoting scar formation. TGF-β1 and TGF-β2 are both increased during wound healing in the adult and lead to increased ECM production and inflammatory cell infiltration. TGF-β1 also has been shown to decrease matrix metalloproteinase (MMP) expression while increasing the expression of natural MMP inhibitors. The relative proportion of TGF-β3 to TGF-β1 also appears to be an important regulator of scar formation. In scarless fetal wounds, TGF-β3 expression is increased while TGF-β1 remains constant, and in scarring wounds TGF-β1 levels increase while TGF-β3 levels decrease.

In one embodiment, the invention features administration of compounds that modulate the TGF-β1, TGF-β2, or TGF-β3 signaling pathways including the related intracellular signaling cascade proteins, such as SMADs. Modulation of molecular signaling in embryonic and adult skin are commonly mediated through the TGFβ-SMAD pathway. One of the major downstream pathways is the synthesis of collagen 1, the primary collagen in adult dermis. The relative amount of TGF-β1 and TGF-β3 has been linked to whether or not a scar is produced in response to wound healing.

Compounds useful for the modulation of the TGFβ-SMAD pathways in conjunction with reepithelialization include, without limitation: Eptotermin alfa, Noggin, bone morphogenetic protein activators (Curis/Ortho Biotech), Transforming growth factor-beta-3, Transforming growth factor-beta-1, Transforming growth factor-alpha, Cetermin, Tamoxifen methiodide, Decorin, Kahalalide F, Anti-TGF-beta monoclonal antibody 2G7, ADMP 1, Lerdelimumab, Metelimumab, TGF-beta antagonists (GLYCODesign), A 161906, LF 984, Tetrathiomolybdate, Tranilast, GC 1008, SEK 1005, TGF-beta antagonists (Scios), SR2F, Stamulumab, NeuGene antisense compounds (AVI BioPharma), TJN 598, TGF-beta RI kinase inhibitors (Scios), TGF-beta oligonucleotide nanoparticles (NanoDel), TGF-beta type I receptor inhibitors (In2Gen), TG-C, and Mannose 6 phosphate.

Integrin and ECM-Mediated Signaling

In another embodiment, the invention features administration of compounds that modulate ECM and integrin-mediated signaling. Integrins are heterodimeric transmembrane receptors composed of an α and β subunit. The most prominent constitutively expressed integrins in the adult epidermis include α2β1 (collagen receptor), α3β1 (laminin 5 receptor), α6β4 (laminin receptor), and αvβ5 (vitronectin receptor). Additional integrins, namely α5β1 (fibronectin receptor), αvβ6 (fibronectin and tenascin receptor), and α9β1 (tenascin receptor) are expressed in response to skin damage and wound healing. In normal skin, integrins are primarily expressed in the basal layer and the hair follicle outer root sheath. Interfollicular and hair follicle stem cells are also known to express highest levels of β1 integrin, a molecular signature that is often used to identify and enrich epithelial stem cells.

Compounds useful for the modulation of the integrin-mediated signaling pathways in conjunction with reepithelialization include, without limitation, Applaggin, Kistrin, RO 435054, MK 852, G 4120, SC 49992, TP 9201, Eptifibatide, Tirofiban, Anti-CD 18 monoclonal antibody, Abciximab, Anti-VLA-4 monoclonal antibody PS/2, Lefradafiban, SKF 107260, DU 728, Lamifiban, CGH 400, SC 52012, GR 91669, SKF 106760, Tetrafibricin, Xemilofiban, Lotrafiban, SB 208651, L 703014, MEDI 522, RWJ 50042, Halystatin, C 6822, SDZ GPI 562, TAK 029, SB 1, L 709780, Fradafiban, SB 6, GR 83895, YM 207, BIBW 98, RG 13965, EF 5077, YM 337, Contortrostatin, RWJ 50228, DMP 757, Rovelizumab, SB 207448, SC 56929, L 734217, Disagregin, G 7453, RO 438857, G 5598, RPR 110173, S 1197, ZD 2486, S 1762, FK 633, CY 9652, RO 443888, Sibrafiban, Natalizumab, Roxifiban, XR 300, NSL 9403, L 748415, ME 3277, P 246, TBC 772, RWJ 50271, SC 56631, TRM 147, PS 028, Orbofiban, Alnidofibatide, USB IPA 1302, Monoclonal antibody PMA5, Monoclonal antibody AZ1, MA 16N7C2, RP 431, SB 223245, L 703801, DMP 802, BIO 1050, BIO 1272, L 738167, SR 121566, XU 063, SR 121787, MS 180, MS 28168, ME 3229, integrin antagonists (Integra LifeSciences), Alpha D modulator, Cilengitide, ZD 7349, MLN 0002, T 250, SB 236392, Doxorubicin peptide conjugate, XR 299, integrin antagonists (Celltech), SB 265123, XV 454, MLN 2201, L 734115, SH 306, Cromafiban, TS 963, TS 943, Accutin, Elarofiban, UR 12947, Gantofiban, GR 233548, SM 20302, alphaV-beta3 receptor antagonists (Shire), NSL 96184, SC 68448, FR 158999, S 137, SM 256, integrin antagonists (SIDR), XJ 735, SQ 885, UR 3216, TR 9109, TR 14035, TR 14531, CP 4632, SC 72115, XU 065, VLA-4 antagonists (Biogen/Merck), CT 5219, SB 273005, L 750034, VLA-4 antagonists (Elan/Wyeth), CP 4685, TBC 3486, TBC 3342, ME 3230, RBx 4638, XT 199, VO 514, SB 267268, IVL 745, AR 0510, AR 0598, LFA-1 antagonists (ICOS), integrin receptor antagonists (Johnson & Johnson), ER 68203, Anti-VLA-4 monoclonal antibody HP1/2, Anti-VLA-4 monoclonal antibody TA-2, Anti-VLA-4 monoclonal antibody R1-2, S 787, CT 747, CT 757, CT 767, L 806978, integrin antagonists (Merck & Co), SC 65811, SJ 874, TBC 4257, IC 747, Integrin antagonist (Bayer), VCAM/VLA-4 antagonists (Wyeth, Kaken), S 247, BIRT 0377, VLA-1 inhibitor (Biogen Idec), RP 593, HMR 1794, TAK 024, Integrin antagonists (Sigma Tau), 559090, vitronectin receptor antagonists (Uriach), VLA-4 antagonists (Uriach), Valategrast, R 1295, integrin antagonists (Targesome), alpha-6 integrin antagonists (Dyax), biologically active linear polysaccharides (BioTie Therapies), TBC 4746, LFA-1 antagonists (Tanabe Seiyaku), RBx 7796, Volociximab, CDP 323, F 200, T 0047, CNTO 95, alpha 2 beta 1 integrin inhibitors (BioTie Therapies), E 7820, BIO 5192, PS 460644, DW 908e, integrin inhibitors (Jerini), integrin avBeta3 inhibitors (Nuevolution), R 1541, Lymphocyte function-associated antigen-1 antagonist (Bristol-Myers Squibb), LFA-1 antagonists (Boehringer Ingelheim), TBC 3804, anti-alpha-5 beta-1 integrin antibody (Pfizer), integrin receptor antagonists (Johnson & Johnson), anti-alpha-v beta-6 monoclonal antibodies (Biogen Idec), and alpha 4 integrin antagonists (Elan).

Insulin Growth Factor

In one embodiment, the invention features administration of compounds that modulate the insulin growth factor (IGF) pathway. IGF was recently shown to play a role as a mitogenic modulator of human keratinocytes derived from a keloid-like scar. The bioavailability of IGF in the wound healing environment is partially regulated by the relative levels of IGF-binding proteins to IGF receptors.

Compounds useful for the modulation of the insulin growth factor pathways in conjunction with reepithelialization include, without limitation: Mecasermin, rinfabate, Insulin-like growth factor-II, CEP 903, INX 4437, 486-STOP, carbohydrate-based anti-inflammatories (Praxis/Fairchild), MZ 471, MZ 5156, IGF-1 receptor inhibitors (Telik), HF 0299, EN 122002, IGF-related antagonists (Novo Nordisk/DGI BioTechnologies), NBI 31772, Pasireotide, Rinfabate, OGX 225, mono-specific IGFBP inhibitors (OncoGeneX), IMC A12, IGF-1R kinase inhibitors (Novartis), anti-IGF-1R antibody (Schering Plough), CP 751871, ATL 1101, anti-IGF-1 monoclonal antibody (Pierre Fabre Medicament/Merck), INSM 18, AVE 1642, insulin-like growth factor-1 receptor antagonists (Insmed), insulin-like growth factor therapeutics (Chlorogen), AMG 479, and insulin-like growth factor binding protein 1 (Amgen).

Cytokine Signaling

Interleukins and other cytokines are also important regulators of scar formation. IL-6 and IL-8 are responsible for inflammatory cell migration and activation in response to wounding. IL-10 is an anti-inflammatory cytokine that attenuates the inflammatory response and is thought to contribute to scarless wound healing.

In yet another embodiment, the invention features administration of compounds that modulate cytokine signaling and inflammation. Pro-inflammatory cytokines, including IL-1, TNF-α, IL-6, IL-8, and interferon γ and α, among others, are up-regulated in response to skin damage. Modulation of cytokines are thought to contribute the relative ability of the skin to form a scar in response to injury. The expression of the genes that code for the proinflammatory cytokines is upregulated in response to via nuclear factor kappaB (NF-kappaB) and AP-1, well known proinflammatory transcription factors. Therefore, stimuli that induce up-regulation of the NF-kappaB pathway contribute to the alteration of the levels of proinflammatory cytokines and therefore scar formation.

Compounds useful for the modulation of cytokine and growth factor signaling in conjunction with reepithelialization include, without limitation, Imiquimod/Avara, IL-1alpha, parthenolide, magnolia extract, magnolol, Prasterone, Iguratimod, Suplatast tosilate, Bindarit, Liarozole, UK 122802, ONO 4007, Stiripentol, DUP 983, DUP 630, DMXAA, ICZ, FPP 33, PP 33, Mesoporphyrin, Semapimod, A 802715, Pirfenidone, Sho-seiryu-to, FR 167653, Pentoxifylline, Iboctadekin, Pimecrolimus, Temsirolimus, HEP 689, R 116010, Tadekinig alfa, Prasterone, PB 007, anti-interleukin-18 monoclonal antibodies (CAT), ISIS 104838, Delmitide, P450RAI inhibitors (Cytochroma), ZNC 2381, R 115866, CLX 0921, Thymosin beta-4, M 50367, JTE 607, Licochalcone A, vitamin D signal amplifiers (Cytochroma), TS 011, CF 101, Prasterone phosphocholine, Y 39041, RDP 58 analogues (Genzyme/Synt:em), NPI 1302a-3, AVI 4557, Susalimod, p38 MAP kinase inhibitors (Uriach/Organon), MT 201, interleukin-4/5 secretion inhibitors (Fournier/Zambon), LMP 160, LMP 420, PLR 14, AD-GL0001, CLX 090717, CLX 090502, CRX 102, Ciclosporin, ABT 325, IMS, K 832, CC 10004, Interleukin 6 inhibitor (Y's Therapeutics), YSTH2, CR1 (Nuada), CRX 119, CRX 139, Golimumab, Rambazole, cytokine receptor antagonists (Trillium Therapeutics), high mobility group box chromosomal protein-1 inhibitors (Nautilus/Creabilis), CYT 007, TNFQb, QR 440, CTA 018, K 412, AN 0128, CRX 170, CRx 140, CRX 150, RC 8800, tumour necrosis factor gene therapy (Onc Bio), Recombinant IL-18 binding protein, HMPL 004, tpl2 kinase inhibitors, SPC 839, RTA 401, MPC 7869, INDRA compounds (Active Biotech), APC 0576, NF-kappa-B Decoy oligonucleotide (Anesiva), BG 12, NF-kappa B/IKK2 inhibitors (Uriach), Antisense oligonucleotide NF-KappaB-p65 (Serono/InDex Pharmaceuticals), I-kappa B kinase inhibitors (Millennium Pharmaceuticals), NeuGene antisense compounds (AVI BioPharma), SIM 916, liposomal calagualine (Plantacor), NF-kappa B inhibitors (Serenex), SIM 688, NF-kappa B inhibitors (Scottish Biomedical), NFkappaB pathway inhibitors (4SC), Synthetic triterpenoids (Reata Pharmaceuticals), and RTA 402.

Growth Factor Signaling

Platelet-derived growth factor (PDGF) and members of the fibroblast growth factor (FGF) family have also been implicated as pro-fibrotic cytokines. Expression of these growth factors is transient in embryonic scarless wounds, but more prolonged in wounds in the adult skin. Vascular endothelial growth factor (VEGF) is upregulated during scarless wound healing, but remains unchanged in adult wounds. This observation implies that differential changes in angiogenesis can modulate the rate and nature of the wound healing response.

In yet another embodiment, the invention features administration of compounds that modulate growth factor signaling. Numerous growth factors, including but not limited to members of the fibroblast growth factor (FGF) family (including keratinocytes growth factor), hepatocyte growth factor, vascular endothelial growth factor (VEGF), connective tissue growth factor (CTGF), and platelet-derived growth factor (PDGF), play a role in scar formation. For example, PDGF regulates the production of pro-inflammatory and pro-fibriotic cytokines as a part of the natural wound healing response.

Compounds useful for modulating growth factor signaling in conjunction with reepithelialization include, without limitation: SNK 863, 11A8 SAP, PD 145709, U 86983, Fibroblast growth factor, Anti-PDGF/bFGF sheep monoclonal antibody, Fibroblast growth factor/hyaluronan, Trafermin, Repifermin, SU 4984, SU 5402, SU 6668, RG 8803, Sibrotuzumab, SU 9803, SU 9902, bFGF receptor antagonists (Praecis), Tetrathiomolybdate, Tranilast, VEGF and FGF receptor inhibitors (Johnson & Johnson Pharmaceutical Research and Development, LLC), TMPP, fibroblast growth factor receptor HuCAL antibodies (MorphoSys/ProChon Biotech), Anti-FAP monoclonal antibody F19-I-131, Talabostat, TBC 1635, fibroblast growth factor antagonists (Encysive), MOR 201, Fibroblast growth factor-18, TKI 258, SSR 128129, Fibroblast growth factor 1, fibroblast growth factor receptor inhibitors (Kirin Brewery), BIBF 1120, XL 999, BMS 582664, FGLL, fibroblast growth factor receptor-3 antibodies (Progenika Biopharma), fibroblast growth factor receptor inhibitors (Astex Therapeutics), fibroblast growth factor receptor monoclonal antibodies (ImClone), Squalamine, Bevacizumab, Semaxanib, RPI 4610, Anti-VEGF sheep monoclonal antibody (KS Biomedix Holdings), Pegaptanib, AMG 706, Ranibizumab, Sorafenib, CP 547632, SU 9803, SU 9902, Vatalanib, MV 833, NM 3, IMC 1C11, Vandetanib, Tetrathiomolybdate, Monoclonal antibody 2C3, EG 004, VEGF Trap, TMPP, KDR kinase inhibitors (Merck), anti-VEGFR monoclonal antibodies (ImClone), SP 5.210C, ABT 828, GFB 116, AZD 2171, VEGF immunotherapeutic (Protherics), CDP 791, CEP 7055, vascular endothelial growth factor inhibitors (Encysive), lipocalin derivatives (Pieris), Vascular endothelial growth factor-2 monoclonal antibody, KRN 633, Axitinib, Sunitinib, TKI 258, E 7080, KRN 951, Anti-FLT-1 monoclonal antibody, Bevasiranib, HYB 220, Veglin, XL 844, CX 3543, XL 647, VEGF-B antagonists (Zenyth Therapeutics), anti-VEGF therapeutics (Pharmexa), Vascular endothelial growth factor (Genix), IMC 1121B, BAY 579352, BIBF 1120, XL 999, XL 184, AG 13958, SU 14813, SB 509, CT 322, OSI 930, XL 880, BMS 582664, ZK 304709, VEGF receptor kinase inhibitors (Affymax), KDR kinase inhibitors (Wyeth), XL 820, VEGFR2 inhibitors (ChemDiv), CHIR 265, KDR kinase inhibitors (Amgen), VEGF antagonists (Vegenics), PTC 299, VEGF receptor 2 antagonists (Astellas Pharma), anti-neuropilin monoclonal antibodies (ImClone), MC 18F1, VEGF antagonists (Novagali Pharma), long-acting VEGF antagonists (Bolder BioTechnology), Dextran sulfate, Platelet-derived growth factor, SCH 13929, Melatonin, Imatinib, SU 102, AG 1296, AG 1295, PD 170262, Becaplermin, Anti-PDGF/bFGF sheep monoclonal antibody, CDP 860, SU 6668, AMG 706, Sorafenib, KI 6783, RPR 101511A, KN 1022, CT 52923, CP 868596, GFB 111, Axitinib, Sunitinib, Zvegf3, CR 002, GEM 21S, PDGFR tyrosine kinase inhibitors (Johnson & Johnson), tyrosine kinase inhibitors (SuperGen), PDGF inhibitors (Archemix), BAY 579352, BIBF 1120, anti-PDGF receptor alpha and beta antibodies (ImClone Systems), XL 999, ZK 304709, XL 820, FG 3019, and connective tissue growth factor inhibitors (FibroGen/Sankyo).

Matrix Metalloproteinases

In yet another embodiment, the invention features administration of compounds that modulate matrix metalloproteinase (MMP) activity in the skin. The MMP family is composed of 28 members of metal dependent enzymes that breakdown different extracellular matrix (ECM) components in the body. MMP-1, −3, and −9 are responsible for degrading collagen in human dermis and are up-regulated in response to injury.

Compounds useful for reducing MMP activity in conjunction with reepithelialization include, without limitation: zinc chelators, iron chelators, doxycycline, marimastat, trocade, TIMP-1, TIMP-2, TIMP-3, TIMP-4, RO 314724, Ilomastat, Incyclinide, D 1927, SE 205, MMP inhibitors (Millennium), macrophage metalloelastase inhibitors (Novartis), PCK 3145, BB 2827, Apratastat, ONO 4817, matrix metalloproteinase inhibitors (Procter & Gamble), ABT 518, SC 77964, SC 276, matrix metalloproteinase inhibitors (Pfizer), SI 27, MPC 2130, GW 3333, matrix metalloproteinase inhibitors (Cengent Therapeutics/De Novo, LEO Pharma, Shionogi), RO 282653, S 3536, MMP-12 inhibitor (Serono), TMI 1, dual tumour necrosis factor/matrix metalloproteinase inhibitors (Roche), protease inhibitors (B iopharmacopae), matrix metalloproteinase-13 inhibitors (Alantos, Wyeth), and matrix metalloproteinase antibodies (Dyax).

Pharmaceutical Formulations

The invention features methods of treating skin conditions by treating skin undergoing reepithelialization with a compound that modulates pathways involved in aging-related skin conditions, pigmentation disorders, acne, and scar formation (e.g., those compounds described above).

The administration of a compound of the invention may be by any suitable means. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the invention may be formulated in a variety of ways that are known in the art.

Controlled and/or Extended Release Formulations

Administration of any one of the compounds of this invention in which the active agent is formulated for controlled and/or extended release, is useful, e.g., when agent has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to maximize the contribution of each agent, when used together, to an amount that is therapeutically effective to treat a skin condition selected from an aging related skin condition, a pigmentation disorder, acne, and scar formation. Accordingly, a sustained release formulation may be used to avoid frequent dosing that may be required in order to sustain the plasma levels of both agents at a therapeutic level. For example, in preferable oral compositions of the invention, half-life and mean residency times from ten to twenty hours for the agents of the invention are observed.

Many strategies can be pursued to obtain controlled and/or extended release in which the rate of release exceeds the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes. The release mechanism can be controlled such that the active agent is released at periodic intervals.

Controlled and/or extended release formulations may include a degradable or nondegradable polymer, hydrogel, organogel, or other physical construct that modifies the bioabsorption, half-life or biodegradation of the agent. The controlled and/or extended release formulation can be a material that is painted or otherwise applied onto the afflicted site, either internally or externally. In one example, the invention provides a biodegradable bolus or implant that is surgically inserted at or near a site of interest.

Hydrogels can be used in controlled release formulations for any of the active agents of this invention. Such polymers are formed from macromers with a polymerizable, non-degradable region that is separated by at least one degradable region. For example, the water soluble, non-degradable, region can form the central core of the macromer and have at least two degradable regions which are attached to the core, such that upon degradation, the non-degradable regions (in particular a polymerized gel) are separated, as described in U.S. Pat. No. 5,626,863. Hydrogels can include acrylates, which can be readily polymerized by several initiating systems such as eosin dye, ultraviolet or visible light. Hydrogels can also include polyethylene glycols (PEGs), which are highly hydrophilic and biocompatible. Hydrogels can also include oligoglycolic acid, which is a poly(α-hydroxy acid) that can be readily degraded by hydrolysis of the ester linkage into glycolic acid, a nontoxic metabolite. Other chain extensions can include polylactic acid, polycaprolactone, polyorthoesters, polyanhydrides, and polypeptides. The entire network can be gelled into a biodegradable network that can be used to entrap and homogeneously disperse various active agents of the invention for delivery at a controlled rate.

Chitosan and mixtures of chitosan with carboxymethylcellulose sodium (CMC-Na) have been used as vehicles for the sustained release of drugs, e.g., as described by Inouye et al., Drug Design and Delivery 1: 297-305, 1987. Mixtures of the active agent, when compressed under 200 kg/cm², form a tablet from which the active agent is slowly released upon administration to a subject. The release profile can be changed by varying the ratios of chitosan, CMC-Na, and active agent(s). The tablets can also contain other additives, including lactose, CaHPO₄ dihydrate, sucrose, crystalline cellulose, or croscarmellose sodium. Several examples are given in Table 1.

TABLE 1

| Materials | Tablet components (mg) | | | | | | | | | | | |
|---|---|---|---|---|---|---|---|---|---|---|---|---|
| Active agent | 20 | 20 | 20 | 20 | 20 | 20 | 20 | 20 | 20 | 20 | 20 | 20 |
| Chitosan | 10 | 10 | 10 | 10 | 10 | 20 | 3.3 | 20 | 3.3 | 70 | 40 | 28 |
| Lactose | | 110 | | | | 220 | 36.7 | | | | | |
| CMC-Na | 60 | 60 | 60 | 60 | 60 | 120 | 20 | 120 | 20 | | 30 | 42 |
| CaHPO₄*2H₂O | | | 110 | | | | | 220 | 36.7 | 110 | 110 | 110 |
| Sucrose | 110 | | | | | | | | | | | |
| Crystalline | | | | | 110 | | | | | | | |
| Cellulose | | | | | | | | | | | | |
| Croscarmellose | | | | 110 | | | | | | | | |
| Na | | | | | | | | | | | | |

Baichwal, in U.S. Pat. No. 6,245,356, describes sustained release oral solid dosage forms that includes agglomerated particles of a therapeutically active medicament in amorphous form, a gelling agent, an ionizable gel strength enhancing agent and an inert diluent. The gelling agent can be a mixture of a xanthan gum and a locust bean gum capable of cross-linking with the xanthan gum when the gums are exposed to an environmental fluid. Preferably, the ionizable gel enhancing agent acts to enhance the strength of cross-linking between the xanthan gum and the locust bean gum and thereby prolonging the release of the medicament component of the formulation. In addition to xanthan gum and locust bean gum, acceptable gelling agents that may also be used include those gelling agents well known in the art. Examples include naturally occurring or modified naturally occurring gums such as alginates, carrageenan, pectin, guar gum, modified starch, hydroxypropylmethylcellulose, methylcellulose, and other cellulosic materials or polymers, such as, for example, sodium carboxymethylcellulose and hydroxypropyl cellulose, and mixtures of the foregoing.

In another formulation useful for the active agents of the invention, Baichwal and Staniforth, in U.S. Pat. No. 5,135,757, describe a free-flowing slow release granulation for use as a pharmaceutical excipient that includes about 20-70% or more by weight of a hydrophilic material that includes a heteropolysaccharide (such as, for example, xanthan gum or a derivative thereof) and a polysaccharide material capable of cross-linking the heteropolysaccharide (such as, for example, galactomannans, and most preferably locust bean gum) in the presence of aqueous solutions, and about 30-80% by weight of an inert pharmaceutical filler (such as, for example, lactose, dextrose, sucrose, sorbitol, xylitol, fructose or mixtures thereof). After mixing the excipient with an active agent of the invention, the mixture is directly compressed into solid dosage forms such as tablets. The tablets thus formed slowly release the medicament when ingested and exposed to gastric fluids. By varying the amount of excipient relative to the medicament, a slow release profile can be attained.

In another formulation useful for the active agent of the invention, Shell, in U.S. Pat. No. 5,007,790, describes sustained-release oral drug-dosage forms that release a drug in solution at a rate controlled by the solubility of the drug. The dosage form comprises a tablet or capsule that includes a plurality of particles of a dispersion of a limited solubility drug (such as, for example, prednisolone, or any other agent useful in the present invention) in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug, and leach it from the particles, assuring that drug reaches the stomach in the solution state, which is generally better-tolerated by the stomach than solid-state drug. The programmed eventual dissolution of the polymer depends upon the nature of the polymer and the degree of crosslinking. The polymer is nonfibrillar and substantially water-soluble in its uncrosslinked state, and the degree of crosslinking is sufficient to enable the polymer to remain insoluble for the desired time period, normally at least from about four hours to eight hours or even twelve hours, with the choice depending upon the drug incorporated and the medical treatment involved. Examples of suitable crosslinked polymers that may be used in the invention are gelatin, albumin, sodium alginate, carboxymethyl cellulose, polyvinyl alcohol, and chitin. Depending upon the polymer, crosslinking may be achieved by thermal or radiation treatment or through the use of crosslinking agents such as aldehydes, polyamino acids, metal ions and the like.

Silicone microspheres for pH-controlled gastrointestinal drug delivery that are useful in the formulation of any of the active agents of the invention have been described by Carelli et al., Int. J. Pharmaceutics 179: 73-83, 1999. The microspheres so described are pH-sensitive semi-interpenetrating polymer hydrogels made of varying proportions of poly(methacrylic acid-co-methylmethacrylate) (Eudragit L100 or Eudragit S100) and crosslinked polyethylene glycol 8000 that are encapsulated into silicone microspheres in the 500-1000 μm size range.

Slow-release formulations may include a coating that is not readily water-soluble but is slowly attacked and removed by water, or through which water can slowly permeate. Thus, for example, an active agent of the invention can be spray-coated with a solution of a binder under continuously fluidizing conditions, such as described by Kitamori et al. (U.S. Pat. No. 4,036,948). Water-soluble binders include pregelatinized starch (e.g., pregelatinized corn starch, pregelatinized white potato starch), pregelatinized modified starch, water-soluble celluloses (e.g. hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum and gelatin, and organic solvent-soluble binders, such as cellulose derivatives (e.g., cellulose acetate phthalate, hydroxypropylmethyl-cellulose phthalate, ethylcellulose).

Yet another form of sustained release agents can be prepared by microencapsulation of agent particles in membranes which act as microdialysis cells. In such a formulation, gastric fluid permeates the microcapsule walls and swells the microcapsule, allowing the active agent(s) to dialyze out (see, e.g., Tsuei et al., U.S. Pat. No. 5,589,194). One commercially available sustained-release system of this kind consists of microcapsules having membranes of acacia gum/gelatine/ethyl alcohol. This product is available from Eurand Limited (France) under the trade name Diffucaps™. Microcapsules so formulated might be carried in a conventional gelatine capsule or tabletted.

Other extended-release formulation examples are described in U.S. Pat. No. 5,422,123. Thus, a system for the controlled release of an active substance may include (a) a deposit-core comprising an effective amount of the active substance and having defined geometric form, and (b) a support-platform applied to the deposit-core, wherein the deposit-core contains at least the active substance, and at least one member selected from the group consisting of (1) a polymeric material which swells on contact with water or aqueous liquids and a gellable polymeric material wherein the ratio of the swellable polymeric material to the gellable polymeric material is in the range 1:9 to 9:1, and (2) a single polymeric material having both swelling and gelling properties, and wherein the support-platform is an elastic support, applied to said deposit-core so that it partially covers the surface of the deposit-core and follows changes due to hydration of the deposit-core and is slowly soluble and/or slowly gellable in aqueous fluids. The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support-platform, for example about 15 to 20%. Binder(s), hydrophilic agent(s) and hydrophobic agent(s) typically total up to about 50% by weight of the support-platform, for example about 40 to 50%.

Formation of a drug-cyclodextrin complex can modify the drug's solubility, dissolution rate, bioavailability, and/or stability properties.

Polymeric cyclodextrins have also been prepared, as described in U.S. patent application Ser. Nos. 10/021,294 and 10/021,312. The cyclodextrin polymers so formed can be useful for active agents of the present invention. These multifunctional polymeric cyclodextrins are commercially available from Insert Therapeutics, Inc., Pasadena, Calif.

As an alternative to direct complexation with agents, cyclodextrins may be used as an auxiliary additive, e.g. as a carrier, diluent or solubiliser. Formulations that include cyclodextrins and other active agents of the present invention can be prepared by methods similar to the preparations of the cyclodextrin formulations described herein.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Thus, for compositions adapted for oral use, an oral vehicle (e.g., a capsule) containing from between 0.01% to 25% (w/w) active agent. The capsule can be taken one to four times daily, or as needed.

Topical Formulations

In the methods of the invention, compounds of the invention can be delivered to the skin in a topical formulation. Topical formulations include, without limitation, creams, lotions, gels, sticks, ointments, sprays, foams, patches, aerosols, wound dressings, and drops. The formulations can be administered, for example, using a metered dose spray applicator, a micro-needle, iontophoresis, ultrasound penetration enhancement, electroporation, nano/micro-injection, sponge, or by applying and spreading the formulation by hand.

Any conventional pharmacologically and cosmetically acceptable vehicles may be used. For example, compounds may be administered in liposomal formulations that allow the biologically active compounds to enter the skin. Such liposomal formulations are described in, for example, U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. No. 4,877,805 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

The formulations can include various conventional colorants, fragrances, thickeners (e.g., xanthan gum), preservatives, humectants, emollients (e.g., hydrocarbon oils, waxes, or silicones), demulcents, emulsifying excipients, dispersants, penetration enhancers, plasticizing agents, preservatives, stabilizers, demulsifiers, wetting agents, emulsifiers, moisturizers, astringents, deodorants, and the like can be added to provide additional benefits and improve the feel and/or appearance of the topical preparation.

The topical formulations of the invention will typically have a pH of between 5.5 and 8.5 and include from about 0.000001% to 10% (w/v), desirably 0.001% to 0.1% (w/v), of the compounds of the invention.

Antioxidants

The formulations of the invention can also contain one or more antioxidants. Useful antioxidants include, without limitation, thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and malic acid, humic acid, bile acid, bile extracts, bilirubin, biliverdin, EDTA, EGTA, and DTPA), vitamins (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and combinations thereof.

Antioxidants that may be incorporated into the formulations of the invention include natural antioxidants prepared from plant extracts, such as extracts from aloe vera; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof.

The total amount of antioxidant included in the formulations can be from 0.001% to 3% by weight, preferably 0.01% to 1% by weight, in particular 0.05% to 0.5% by weight, based on the total weight of the formulation.

Emulsifying Excipients

Formulations of the invention can further include one or more emulsifying excipients. Emulsifying excipients that may be used in the formulations of the invention include, without limitation, compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, ionic surfactants, tocopherol esters, and sterol esters. Commercially available examples for each class of excipient are provided below.

Polyethoxylated fatty acids may be used as excipients for the formulations of the invention. Examples of commercially available polyethoxylated fatty acid monoester surfactants include: PEG 4-100 monolaurate (Crodet L series, Croda), PEG 4-100 monooleate (Crodet O series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-1EX, Nikko, and Coster K1, Condea), PEG-2 stearate (Nikkol MYS-2, Nikko), PEG-2 oleate (Nikkol MYO-2, Nikko), PEG-4 laurate (Mapeg® 200 ML, PPG), PEG-4 oleate (Mapeg® 200 MO, PPG), PEG-4 stearate (Kessco® PEG 200 MS, Stepan), PEG-5 stearate (Nikkol TMGS-5, Nikko), PEG-5 oleate (Nikkol TMGO-5, Nikko), PEG-6 oleate (Algon OL 60, Auschem SpA), PEG-7 oleate (Algon OL 70, Auschem SpA), PEG-6 laurate (Kessco® PEG300 ML, Stepan), PEG-7 laurate (Lauridac 7, Condea), PEG-6 stearate (Kessco® PEG300 MS, Stepan), PEG-8 laurate (Mapeg® 400 ML, PPG), PEG-8 oleate (Mapeg® 400 MO, PPG), PEG-8 stearate (Mapeg® 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco® PEG 600 ML, Stepan), PEG-12 oleate (Kessco® PEG 600 MO, Stepan), PEG-12 ricinoleate (CAS #9004-97-1), PEG-12 stearate (Mapeg® 600 MS, PPG), PEG-15 stearate (Nikkol TMGS-15, Nikko), PEG-15 oleate (Nikkol TMGO-15, Nikko), PEG-20 laurate (Kessco® PEG 1000 ML, Stepan), PEG-20 oleate (Kessco® PEG 1000 MO, Stepan), PEG-20 stearate (Mapeg® 1000 MS, PPG), PEG-25 stearate (Nikkol MYS-25, Nikko), PEG-32 laurate (Kessco® PEG 1540 ML, Stepan), PEG-32 oleate (Kessco® PEG 1540 MO, Stepan), PEG-32 stearate (Kessco® PEG 1540 MS, Stepan), PEG-30 stearate (Myrj 51), PEG-40 laurate (Crodet L40, Croda), PEG-40 oleate (Crodet 040, Croda), PEG-40 stearate (Emerest® 2715, Henkel), PEG-45 stearate (Nikkol MYS-45, Nikko), PEG-50 stearate (Myrj 53), PEG-55 stearate (Nikkol MYS-55, Nikko), PEG-100 oleate (Crodet O-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf.). Formulations of the invention may include one or more of the polyethoxylated fatty acids above.

Polyethylene glycol fatty acid diesters may be used as excipients for the formulations of the invention. Examples of commercially available polyethylene glycol fatty acid diesters include: PEG-4 dilaurate (Mapeg® 200 DL, PPG), PEG-4 dioleate (Mapeg® 200 DO, PPG), PEG-4 distearate (Kessco® 200 DS, Stepan), PEG-6 dilaurate (Kessco® PEG 300 DL, Stepan), PEG-6 dioleate (Kessco® PEG 300 DO, Stepan), PEG-6 distearate (Kessco® PEG 300 DS, Stepan), PEG-8 dilaurate (Mapeg® 400 DL, PPG), PEG-8 dioleate (Mapeg® 400 DO, PPG), PEG-8 distearate (Mapeg® 400 DS, PPG), PEG-10 dipalmitate (Polyaldo 2PKFG), PEG-12 dilaurate (Kessco® PEG 600 DL, Stepan), PEG-12 distearate (Kessco® PEG 600 DS, Stepan), PEG-12 dioleate (Mapeg® 600 DO, PPG), PEG-20 dilaurate (Kessco® PEG 1000 DL, Stepan), PEG-20 dioleate (Kessco® PEG 1000 DO, Stepan), PEG-20 distearate (Kessco® PEG 1000 DS, Stepan), PEG-32 dilaurate (Kessco® PEG 1540 DL, Stepan), PEG-32 dioleate (Kessco® PEG 1540 DO, Stepan), PEG-32 distearate (Kessco® PEG 1540 DS, Stepan), PEG-400 dioleate (Cithrol 4DO series, Croda), and PEG-400 distearate Cithrol 4DS series, Croda). Formulations of the invention may include one or more of the polyethylene glycol fatty acid diesters above.

PEG-fatty acid mono- and di-ester mixtures may be used as excipients for the formulations of the invention. Examples of commercially available PEG-fatty acid mono- and di-ester mixtures include: PEG 4-150 mono, dilaurate (Kessco® PEG 200-6000 mono, Dilaurate, Stepan), PEG 4-150 mono, dioleate (Kessco® PEG 200-6000 mono, Dioleate, Stepan), and PEG 4-150 mono, distearate (Kessco® 200-6000 mono, Distearate, Stepan). Formulations of the invention may include one or more of the PEG-fatty acid mono- and di-ester mixtures above.

Polyethylene glycol glycerol fatty acid esters may be used as excipients for the formulations of the invention. Examples of commercially available polyethylene glycol glycerol fatty acid esters include: PEG-20 glyceryl laurate (Tagat® L, Goldschmidt), PEG-30 glyceryl laurate (Tagat® L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldo® MS-20 KFG, Lonza), PEG-20 glyceryl oleate (Tagat® O, Goldschmidt), and PEG-30 glyceryl oleate (Tagat® O2, Goldschmidt). Formulations of the invention may include one or more of the polyethylene glycol glycerol fatty acid esters above.

Alcohol-oil transesterification products may be used as excipients for the formulations of the invention. Examples of commercially available alcohol-oil transesterification products include: PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PRT 56, Pulcra SA), PEG-60 castor oil (Nikkol CO-60TX, Nikko), PEG-100 castor oil, PEG-200 castor oil (Eumulgin® PRT 200, Pulcra SA), PEG-5 hydrogenated castor oil (Nikkol HCO-5, Nikko), PEG-7 hydrogenated castor oil (Cremophor WO7, BASF), PEG-10 hydrogenated castor oil (Nikkol HCO-10, Nikko), PEG-20 hydrogenated castor oil (Nikkol HCO-20, Nikko), PEG-25 hydrogenated castor oil (Simulsol® 1292, Seppic), PEG-30 hydrogenated castor oil (Nikkol HCO-30, Nikko), PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO-100, Nikko), PEG-6 corn oil (Labrafil® M 2125 CS, Gattefosse), PEG-6 almond oil (Labrafil® M 1966 CS, Gattefosse), PEG-6 apricot kernel oil (Labrafil® M 1944 CS, Gattefosse), PEG-6 olive oil (Labrafil® M 1980 CS, Gattefosse), PEG-6 peanut oil (Labrafil® M 1969 CS, Gattefosse), PEG-6 hydrogenated palm kernel oil (Labrafil® M 2130 BS, Gattefosse), PEG-6 palm kernel oil (Labrafil® M 2130 CS, Gattefosse), PEG-6 triolein (Labrafil® M 2735 CS, Gattefosse), PEG-8 corn oil (Labrafil® WL 2609 BS, Gattefosse), PEG-20 corn glycerides (Crovol M40, Croda), PEG-20 almond glycerides (Crovol A40, Croda), PEG-25 trioleate (TAGAT® TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labrafac® Hydro, Gattefosse), PEG-8 caprylic/capric glycerides (Labrasol, Gattefosse), PEG-6 caprylic/capric glycerides (SOFTIGEN®767, Huls), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol DS, Taiwan Surf.), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nikkol Pentarate 408, Nikko). Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants. Formulations of the invention may include one or more of the alcohol-oil transesterification products above.

Polyglycerized fatty acids may be used as excipients for the formulations of the invention. Examples of commercially available polyglycerized fatty acids include: polyglyceryl-2 stearate (Nikkol DGMS, Nikko), polyglyceryl-2 oleate (Nikkol DGMO, Nikko), polyglyceryl-2 isostearate (Nikkol DGMIS, Nikko), polyglyceryl-3 oleate (Caprol® 3GO, ABITEC), polyglyceryl-4 oleate (Nikkol Tetraglyn 1-O, Nikko), polyglyceryl-4 stearate (Nikkol Tetraglyn 1-S, Nikko), polyglyceryl-6 oleate (Drewpol 6-1-O, Stepan), polyglyceryl-10 laurate (Nikkol Decaglyn 1-L, Nikko), polyglyceryl-10 oleate (Nikkol Decaglyn 1-O, Nikko), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-O, Nikko), polyglyceryl-3 dioleate (Cremophor GO32, BASF), polyglyceryl-3 distearate (Cremophor GS32, BASF), polyglyceryl-4 pentaoleate (Nikkol Tetraglyn 5-O, Nikko), polyglyceryl-6 dioleate (Caprol® 6G20, ABITEC), polyglyceryl-2 dioleate (Nikkol DGDO, Nikko), polyglyceryl-10 trioleate (Nikkol Decaglyn 3-O, Nikko), polyglyceryl-10 pentaoleate (Nikkol Decaglyn 5-O, Nikko), polyglyceryl-10 septaoleate (Nikkol Decaglyn 7-O, Nikko), polyglyceryl-10 tetraoleate (Caprol® 10G4O, ABITEC), polyglyceryl-10 decaisostearate (Nikkol Decaglyn 10-IS, Nikko), polyglyceryl-101 decaoleate (Drewpol 10-10-O, Stepan), polyglyceryl-10 mono, dioleate (Caprol® PGE 860, ABITEC), and polyglyceryl polyricinoleate (Polymuls, Henkel). Formulations of the invention may include one or more of the polyglycerized fatty acids above.

Propylene glycol fatty acid esters may be used as excipients for the formulations of the invention. Examples of commercially available propylene glycol fatty acid esters include: propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-O6, Eastman), propylene glycol dicaprylate dicaprate (Captex® 200, ABITEC), propylene glycol dioctanoate (Captex® 800, ABITEC), propylene glycol caprylate caprate (LABRAFAC PG, Gattefosse), propylene glycol dilaurate, propylene glycol distearate (Kessco® PGDS, Stepan), propylene glycol dicaprylate (Nikkol Sefsol 228, Nikko), and propylene glycol dicaprate (Nikkol PDD, Nikko). Formulations the invention may include one or more of the propylene glycol fatty acid esters above.

Mixtures of propylene glycol esters and glycerol esters may be used as excipients for the formulations of the invention. One preferred mixture is composed of the oleic acid esters of propylene glycol and glycerol (Arlacel 186). Examples of these surfactants include: oleic (ATMOS 300, ARLACEL 186, ICI), stearic (ATMOS 150). Formulations of the invention may include one or more of the mixtures of propylene glycol esters and glycerol esters above.

Mono- and diglycerides may be used as excipients for the formulations of the invention. Examples of commercially available mono- and diglycerides include: monopalmitolein (C 16:1) (Larodan), monoelaidin (C 18:1) (Larodan), monocaproin (C6) (Larodan), monocaprylin (Larodan), monocaprin (Larodan), monolaurin (Larodan), glyceryl monomyristate (C14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen® 701, Huls), glyceryl monolaurate (ALDO® MLD, Lonza), glycerol monopalmitate (Emalex GMS-P, Nihon), glycerol monostearate (Capmul® GMS, ABITEC), glyceryl mono- and dioleate (Capmul® GMO-K, ABITEC), glyceryl palmitic/stearic (CUTINA MD-A, ESTAGEL-G18), glyceryl acetate (Lamegin® EE, Grunau GmbH), glyceryl laurate (Imwitor® 312, Huls), glyceryl citrate/lactate/oleate/linoleate (Imwitor® 375, Huls), glyceryl caprylate (Imwitor® 308, Huls), glyceryl caprylate/caprate (Capmul® MCM, ABITEC), caprylic acid mono- and diglycerides (Imwitor® 988, Huls), caprylic/capric glycerides (Imwitor® 742, Huls), Mono-and diacetylated monoglycerides (Myvacet® 9-45, Eastman), glyceryl monostearate (Aldo® MS, Arlacel 129, ICD, lactic acid esters of mono and diglycerides (LAMEGIN GLP, Henkel), dicaproin (C6) (Larodan), dicaprin (C10) (Larodan), dioctanoin (C8) (Larodan), dimyristin (C14) (Larodan), dipalmitin (C16) (Larodan), distearin (Larodan), glyceryl dilaurate (C12) (Capmul® GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan). Formulations of the invention may include one or more of the mono- and diglycerides above.

Sterol and sterol derivatives may be used as excipients for the formulations of the invention. Examples of commercially available sterol and sterol derivatives include: cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nikkol BPSH-25, Nikko), PEG-5 soyasterol (Nikkol BPS-5, Nikko), PEG-10 soyasterol (Nikkol BPS-10, Nikko), PEG-20 soyasterol (Nikkol BPS-20, Nikko), and PEG-30 soyasterol (Nikkol BPS-30, Nikko). Formulations of the invention may include one or more of the sterol and sterol derivatives above.

Polyethylene glycol sorbitan fatty acid esters may be used as excipients for the formulations of the inveniton. Examples of commercially available polyethylene glycol sorbitan fatty acid esters include: PEG-10 sorbitan laurate (Liposorb L-10, Lipo Chem.), PEG-20 sorbitan monolaurate (Tween® 20, Atlas/ICI), PEG-4 sorbitan monolaurate (Tween® 21, Atlas/ICI), PEG-80 sorbitan monolaurate (Hodag PSML-80, Calgene), PEG-6 sorbitan monolaurate (Nikkol GL-1, Nikko), PEG-20 sorbitan monopalmitate (Tween® 40, Atlas/ICI), PEG-20 sorbitan monostearate (Tween® 60, Atlas/ICI), PEG-4 sorbitan monostearate (Tween® 61, Atlas/ICI), PEG-8 sorbitan monostearate (DACOL MSS, Condea), PEG-6 sorbitan monostearate (Nikkol TS 106, Nikko), PEG-20 sorbitan tristearate (Tween® 65, Atlas/ICI), PEG-6 sorbitan tetrastearate (Nikkol GS-6, Nikko), PEG-60 sorbitan tetrastearate (Nikkol GS-460, Nikko), PEG-5 sorbitan monooleate (Tween® 81, Atlas/ICI), PEG-6 sorbitan monooleate (Nikkol TO-106, Nikko), PEG-20 sorbitan monooleate (Tween® 80, Atlas/ICI), PEG-40 sorbitan oleate (Emalex ET 8040, Nihon Emulsion), PEG-20 sorbitan trioleate (Tween® 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween® 120, Atlas/ICI), PEG sorbitol hexaoleate (Atlas G-1086, ICD, polysorbate 80 (Tween® 80, Pharma), polysorbate 85 (Tween® 85, Pharma), polysorbate 20 (Tween® 20, Pharma), polysorbate 40 (Tween® 40, Pharma), polysorbate 60 (Tween® 60, Pharma), and PEG-6 sorbitol hexastearate (Nikkol GS-6, Nikko). Formulations of the invention may include one or more of the polyethylene glycol sorbitan fatty acid esters above.

Polyethylene glycol alkyl ethers may be used as excipients for the formulations of the invention. Examples of commercially available polyethylene glycol alkyl ethers include: PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICD. Formulations of the invention may include one or more of the polyethylene glycol alkyl ethers above.

Sugar esters may be used as excipients for the formulations of the invention. Examples of commercially available sugar esters include: sucrose distearate (SUCRO ESTER 7, Gattefosse), sucrose distearate/monostearate (SUCRO ESTER 11, Gattefosse), sucrose dipalmitate, sucrose monostearate (Crodesta F-160, Croda), sucrose monopalmitate (SUCRO ESTER 15, Gattefosse), and sucrose monolaurate (Saccharose monolaurate 1695, Mitsubisbi-Kasei). Formulations of the invention may include one or more of the sugar esters above.

Polyethylene glycol alkyl phenols may be used as excipients for the formulations of the invention. Examples of commercially available polyethylene glycol alkyl phenols include: PEG-10-100 nonylphenol series (Triton X series, Rohm & Haas) and PEG-15-100 octylphenol ether series (Triton N-series, Rohm & Haas). Formulations of the invention may include one or more of the polyethylene glycol alkyl phenols above.

Polyoxyethylene-polyoxypropylene block copolymers may be used as excipients for the formulations of the invention. These surfactants are available under various trade names, including one or more of Synperonic PE series (ICD, Pluronic® series (BASF), Lutrol (BASF), Supronic, Monolan, Pluracare, and Plurodac. The generic term for these polymers is “poloxamer” (CAS 9003-11-6). These polymers have the formula I:

HO(C₂H₄O)ₐ(C₃H₆O)_{b}(C₂H₄O)ₐH (I)

where “a” and “b” denote the number of polyoxyethylene and polyoxypropylene units, respectively. Formulations of the invention may include one or more of the polyoxyethylene-polyoxypropylene block copolymers above.

Polyoxyethylenes, such as PEG 300, PEG 400, and PEG 600, may be used as excipients for the formulations of the invention.

Sorbitan fatty acid esters may be used as excipients for the formulations of the invention. Examples of commercially sorbitan fatty acid esters include: sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICD, sorbitan tristearate (Span-65, Atlas/ICI), sorbitan monoisostearate (Crill 6, Croda), and sorbitan sesquistearate (Nikkol SS-15, Nikko). Formulations of the invention may include one or more of the sorbitan fatty acid esters above.

Esters of lower alcohols (C2 to C4) and fatty acids (C8 to C18) are suitable surfactants for use in the invention. Examples of these surfactants include: ethyl oleate (Crodamol EO, Croda), isopropyl myristate (Crodamol IPM, Croda), isopropyl palmitate (Crodamol IPP, Croda), ethyl linoleate (Nikkol VF-E, Nikko), and isopropyl linoleate (Nikkol VF-IP, Nikko). Formulations of the invention may include one or more of the lower alcohol fatty acid esters above.

Ionic surfactants may be used as excipients for the formulations of the invention. Examples of useful ionic surfactants include: sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of the invention may include one or more of the ionic surfactants above.

Tocopherol esters and sterol esters, as described in U.S. Pat. Nos. 6,632,443 and 6,191,172, each of which is incorporated herein by reference, may be used as excipients for the formulations of the invention. These tocopherol and sterol esters are described by formula II:

{X—OOC—[(CH₂)ₙ—COO]ₘ}ₚ—Y (II)

wherein X is selected from α-tocopherol, β-tocopherol, γ-tocopherol, δ-tocopherol, cholesterol, 7-dehydrocholesterol, campesterol, sitosterol, ergosterol, and stigmasterol; p is 1 or 2; m is 0 or 1; n is an integer from 0 to 18; and Y is a hydrophilic moiety selected from polyalcohols, polyethers, and derivatives thereof.

The emulsifying excipients present in the formulations of the invention are present in amounts such that the carrier forms uniform dispersion of compounds of the invention. The relative amounts of surfactants required are readily determined by observing the properties of the resultant dispersion, as determined using standard techniques for measuring solubilities. The optical clarity of the aqueous dispersion can be measured using standard quantitative techniques for turbidity assessment. For example, a formulation of the invention can include from 0.001% to 10% by weight, preferably 0.01% to 5% by weight, emulsifying excipient.

Gelling Agents

Formulations of the invention can also contain one or more gelling agents. Useful gelling agents include, without limitation, hydroxyethylcellulose (commercially available as NATROSOL® hydroxyethylcellulose produced by Aqualon), hydroxypropylcellulose (commercially available as KLUCEL® hydroxypropylcellulose produced by Aqualon), cross-linked acrylic acid polymers (such as the commercially available product CARBOPOL® cross linked acrylic acid polymer, produced by Goodrich), MVE/MA decadiene crosspolymer (such as the commercially available product STABILEZE® MVE/MA decadiene crosspolymer, produced by ISP), PVM/MA copolymer (such as the commercially available product GANTREZ® PVM/MA copolymer, produced by ISP), ammonium acrylates/acrylonitrogens (commercially available as HYPAN® ammonium acrylates/acrylonitrogens), carboxymethylcellulose, polyvinylpyrrolidone, carbomer (carboxypolymethylene, CAS 541823-57-9; of which different grades with various molecular weights are commercially available), cetostearyl alcohol, colloidal silicon dioxide, gelatin, guar gum, sodium or calcium carboxymethyl cellulose, hydroxyethyl or hydroxypropyl cellulose, hydroxypropylmethylcellulose, methyl or ethyl cellulose, maltodextrin, polyvinyl alcohol, propylene carbonate, povidone, propylene glycol alginate, alginic acid sodium alginate, sodium starch glycolate, starch, and sucrose. Typically, the gelling agent, when used, is present in an amount between about 0.5% to about 10% by weight of the composition. More particularly, for CARBOPOL® cross linked acrylic acid polymer the preferred compositional weight percent range is between about 2% to about 6%, while for NATROSOL® hydroxyethylcellulose or KLUCEL® hydroxypropylcellulose the preferred range is between about 0.5% to about 4%. Desirably, the compositional weight percent range for STABILEZE® PVM/MA decadiene crosspolymer and HYPAN® ammonium acrylates/acrylonitrogens is between about 1% to about 4%. The preferred compositional weight percent range for polyvinylpyrrolidone is between about 0.5% and about 10%.

Hydrocolloids

Formulations of the invention can contain one or more hydrocolloids. Useful hydrocolloids include, without limitation, Carbopol, including Carbopol 940, carrageenan, agar, xanthan gum, locust bean gum polyglucomannan, and gelatin.

Cross-Linking Agents

Formulations of the invention can contain one or more cross-linking agents to form a chemical bond between the molecules of the polymer to gel the dispersion, forming a solid body. Examples of cross-linking agents for locust bean gum, guar or chemically modified guar are galactose, organic titanate or boric acid. When the hydrocolloid is a polyglucomannan (e.g., Konjak®), borax can be used as a cross-linking agent. When xanthan gum is used, a suitable cross-linker for xanthan gum is mannose. If locust bean gum is used as the principle hydrocolloid, lactose or other suitable oligosaccharide can be used.

Plasticizers

Formulations of the invention can contain one or more plasticizers. Useful plasticizers include, without limitation, alkyl glycols, polyalkylene glycols (e.g., polyethylene glycol and/or polypropylene glycol), benzyl benzoate, chlorobutanol, mineral oil, (CTFA mixture of mineral oils, e.g., Amerchol L-101, Protalan M-16, Protalan M-26), petrolatum (CTFA, mixture of petrolatum, e.g., Amerchol CAB, Forlan 200), lanolin alcohols, sorbitol, triacetin, dibutyl sebacate, diethyl phthalate, glycerine, petrolactam and triethyl citrate.

Other Biologically Active Ingredients

If desired, the formulations of the invention can be combined with additional active ingredients. Desirably, the compounds of the invention and the additional active ingredient or ingredients are formulated together. The amount of an additional active ingredient included will depend on the desired effect and the active ingredient that is selected. In general, the amount of an additional active ingredient varies from about 0.0001% to about 20%, preferably from about 0.01% to about 10%, or even about 0.1% to about 5% by weight.

Other biologically active agents that can be used in the methods, kits, and compositions of the invention include antihistamines, anti-inflammatory agents, retinoids, anti-androgen agents, immunosuppressants, channel openers, antimicrobials, herbs (e.g., saw palmetto), extracts (e.g., Souhakuhi extract), vitamins (e.g., biotin), co-factors, psoralen, anthralin, and antibiotics.

Antihistamines

In certain embodiments, an antihistamine can be used in the compositions, methods, and kits of the invention. Useful antihistamines include, without limitation, Ethanolamines (e.g., bromodiphenhydramine, carbinoxamine, clemastine, dimenhydrinate, diphenhydramine, diphenylpyraline, and doxylamine); Ethylenediamines (e.g., pheniramine, pyrilamine, tripelennamine, and triprolidine); Phenothiazines (e.g., diethazine, ethopropazine, methdilazine, promethazine, thiethylperazine, and trimeprazine); Alkylamines (e.g., acrivastine, brompheniramine, chlorpheniramine, desbrompheniramine, dexchlorpheniramine, pyrrobutamine, and triprolidine); Piperazines (e.g., buclizine, cetirizine, chlorcyclizine, cyclizine, meclizine, hydroxyzine); Piperidines (e.g., astemizole, azatadine, cyproheptadine, desloratadine, fexofenadine, loratadine, ketotifen, olopatadine, phenindamine, and terfenadine); and Atypical antihistamines (e.g., azelastine, levocabastine, methapyrilene, and phenyltoxamine). Both non-sedating and sedating antihistamines may be employed. Non-sedating antihistamines include loratadine and desloratadine. Sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the compositions, methods, and kits of the invention are acrivastine; ahistan; antazoline; astemizole; azelastine; bamipine; bepotastine; bietanautine; brompheniramine; carbinoxamine; cetirizine; cetoxime; chlorocyclizine; chloropyramine; chlorothen; chlorphenoxamine; cinnarizine; clemastine; clobenzepam; clobenztropine; clocinizine; cyclizine; deptropine; dexchlorpheniramine; dexchlorpheniramine maleate; diphenylpyraline; doxepin; ebastine; embramine; emedastine; epinastine; etymemazine hydrochloride; fexofenadine; histapyrrodine; hydroxyzine; isopromethazine; isothipendyl; levocabastine; mebhydroline; mequitazine; methafurylene; methapyrilene; metron; mizolastine; olapatadine; orphenadrine; phenindamine; pheniramine; phenyltoloxamine; p-methyldiphenhydramine; pyrrobutamine; setastine; talastine; terfenadine; thenyldiamine; thiazinamium; thonzylamine hydrochloride; tolpropamine; triprolidine; and tritoqualine.

Antihistamine analogs can be used in the compositions, methods, and kits of the invention. Antihistamine analogs include 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone, 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-1-dibenzo(a,d)cyclohepten-5-ylidene)-tropane.

Other compounds that are suitable for use in the compositions, methods, and kits of the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that can be used in the compositions, methods, and kits of the invention are described in U.S. Pat. Nos. 3,956,296; 4,254,129; 4,254,130; 4,282,233; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Antimicrobial Agents

In certain embodiments, an antimicrobial agent can be used in the compositions, methods, and kits of the invention. Useful antimicrobial agents include, without limitation, benzyl benzoate, benzalkonium chloride, benzoic acid, benzyl alcohol, butylparaben, ethylparaben, methylparaben, propylparaben, camphorated metacresol, camphorated phenol, hexylresorcinol, methylbenzethonium chloride, cetrimide, chlorhexidine, chlorobutanol, chlorocresol, cresol, glycerin, imidurea, phenol, phenoxyethanol, phenylethylalcohol, phenylmercuric acetate, phenylmercuric borate, phenylmercuric nitrate, potassium sorbate, sodium benzoate, sodium proprionate, sorbic acid, and thiomersal.

The antimicrobial can be from about 0.05% to 0.5% by weight of the total composition, except for-camphorated-phenol and camphorated metacresol. For camphorated phenol, the preferred weight percentages are about 8% to 12% camphor and about 3% to 7% phenol. For camphorated metacresol, the preferred weight percentages are about 3% to 12% camphor and about 1% to 4% metacresol.

Anti-Inflammatory Agents

In certain embodiments, an antiinflammtory agent can be used in the compositions, methods, and kits of the invention. Useful antiinflammtory agents include, without limitation, Non-Steroidal Anti-Inflammtory Drugs (NSAIDs) (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), and corticosteroids (e.g., alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide).

Immunosuppressants

In certain embodiments, a nonsteroidal immunosuppressant can be used in the compositions, methods, and kits of the invention. Suitable immunosuppressants include cyclosporine, tacrolimus, rapamycin, everolimus, and pimecrolimus.

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al., Antimicrob. Agents Chemother. 44:143 (2000).

Tacrolimus and tacrolimus analogs are described by Tanaka et al. (J. Am. Chem. Soc., 109:5031 (1987)) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus is the 33-epi-chloro derivative of the macrolactam ascomyin. Pimecrolimus structural and functional analogs are described in U.S. Pat. No. 6,384,073.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Retinoids

In certain embodiments, a retinoid can be used in the compositions, methods, and kits of the invention. Useful retinoids include, without limitation, 13-cis-retinoic acid, adapalene, all-trans-retinoic acid, and etretinate.

Channel Openers

In certain embodiments, a channel opener can be used in the compositions, methods, and kits of the invention. Useful channel openers include, without limitation, minoxidil, diazoxide, and phenytoin.

Anti-Androgens

In certain embodiments, an anti-androgen can be used in the compositions, methods, and kits of the invention. Useful anti-androgens include, without limitation, finasteride, flutamide, diazoxide, 11 alpha-hydroxyprogesterone, ketoconazole, RU58841, dutasteride, fluridil, QLT-7704, and anti-androgen oligonucleotides.

Antibiotics

In certain embodiments, an antibiotic can be used in the compositions, methods, and kits of the invention. Useful antibiotics include, without limitation, penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, and trimethoprim.

Kits

The invention also features kits for the treatment of skin conditions including aging-related skin conditions, pigmentation disorders, acne, and scar formation. In one embodiment, the kits of the invention include a therapeutic compound and instructions for administering the therapeutic compound to skin undergoing reepithelialization. In this embodiment, the kits may also contain a means for inducing reepithelialization in skin. Also in this embodiment, the kits may also contain an additional biologically active compound.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1-176. (canceled)

177. A method of treating or preventing scar formation in a subject, comprising
(a) disrupting the skin of said subject; and
(b) contacting the cells of the skin of said subject with a therapeutic compound,
wherein said therapeutic compound is administered in an amount sufficient to improve said scar formation.

178. The method of claim 177, wherein said disrupting produces skin characterized by an embryonic-like state or a substantial lack of a stratum corneum.

179. The method of claim 177, wherein said disrupting is sub-epidermal.

180. The method of claim 177, wherein said disrupting is dermal.

181. The method of claim 177, wherein said disrupting does not result in disturbance to the stratum corneum or upper epidermis.

182. The method of claim 177, wherein said disrupting comprises using a device.

183. The method of claim 182, wherein said device is selected from the group consisting of sandpaper, a felt wheel, ultrasound, dermabrasion, microdermabrasion, a laser, a supersonically accelerated mixture of saline and oxygen, tape-stripping, and peels.

184. The method of claim 183, wherein said device is a laser.

185. The method of claim 184, wherein said laser is a fractional laser, a CO₂ laser, or an excimer laser.

186. The method of claim 182, wherein said device is selected from the group consisting of pumice pads, Scotch-Brite pads, and microneedles.

187. The method of claim 177, wherein said disrupting comprises using a chemical.

188. The method of claim 187, wherein said chemical is selected from the group consisting of phenol, trichloracetic acid, and ascorbic acid.

189. The method of claim 187, wherein said chemical is a protease selected from the group consisting of of papain, bromelain, stratum corneum chymotryptic enzyme, trypsin, dispase, and thermolysin.

190. The method of claim 177, wherein said disrupting comprises using electromagnetic radiation.

191. The method of claim 177, wherein said disrupting comprises using electroporation.

192. The method of claim 177, wherein said disrupting comprises using acoustic radiation.

193. The method of claim 177, wherein said therapeutic compound is a compound that modulates a pathway selected from the group consisting of TGF-β signaling, integrin and ECM-mediated signaling, insulin growth factor signaling, cytokine signaling, growth factor signaling, and matrix metalloproteinase signaling.

194. The method of claim 177, wherein said therapeutic compound is a cell.

195. The method of claim 177, wherein said contacting the cells of said skin with a therapeutic compound is performed 1, 2, 3, 4, 5, 10, 15, 24, or 48 hours after said disrupting.

196. The method of claim 177, wherein said contacting the cells of said skin with a therapeutic compound is performed 1, 2, 3, 4, 5, 6, 7, 10, 14, or 21 days after said disrupting.

197. The method of claim 177, wherein said contacting the cells of said skin with a therapeutic compound is performed before said disrupting.

198. The method of claim 177, wherein said subject is a human.

199. A method of treating an aging related skin condition in a subject, comprising:
(a) disrupting the skin of said subject; and
(b) contacting the cells of said skin with a therapeutic compound,
wherein said therapeutic compound is administered in an amount sufficient to improve said aging related skin condition.

200. A method of treating a pigmentation disorder in a subject, comprising:
(a) disrupting the skin of said subject; and
(b) contacting the cells of said skin with a therapeutic compound,
wherein said therapeutic compound is administered in an amount sufficient to improve said pigmentation disorder.

201. A method of treating acne in a subject, comprising:
(a) disrupting the skin of said subject; and
(b) contacting the cells of said skin with a therapeutic compound,
wherein said therapeutic compound is administered in an amount sufficient to improve said acne.
